(19) United States
(12) Patent Application Publication
(10) Pub. No.: US 2016/0106587 A1
(43) Pub. Date: Apr. 21, 2016

(54) OCULAR GELS OR HYDROGELS AND MICROINJECTORS

(71) Applicant: Incept, LLC, Lexington, MA (US)

(72) Inventors: Peter Jarrett, Lexington, MA (US); Michael J. McGrath, Upton, MA (US); Rami El-Hayek, Norwood, MA (US); Amarpreet S. Sawhney, Lexington, MA (US)

(21) Appl. No.: 14/878,243

(22) Filed: Oct. 8, 2015

Related U.S. Application Data
(60) Provisional application No. 62/064,885, filed on Oct. 16, 2014.

(51) Int. Cl. A61F 9/00; A61K 9/00; A61K 31/573; A61K 9/06; A61M 5/315; A61M 5/19

(52) U.S. Cl. CPC A61F 9/0026; A61M 5/315; A61M 5/19; A61K 31/573; A61K 9/06; A61K 9/0048

(57) ABSTRACT

Devices and materials for treating an ophthalmic pathology affecting an eye of a patient comprising forming, in situ, a continuous cohesive layer of covalently-crosslinked hydrogel at a choroid in the eye, wherein the hydrogel comprises a therapeutic agent that is released into the eye to treat the ophthalmic pathology. An injection device for injection into a tissue, for example an eye, comprising a syringe, and a needle, with the syringe comprising a reservoir in fluid communication with an outlet and a piston for expelling contents of the reservoir from the syringe through the outlet. An embodiment provides for setting or using a force for moving the piston so as to deliver the contents at, for instance, a suprachoroidal location.

### CROSS-REFERENCE TO RELATED APPLICATIONS

This application is claims priority to U.S. Provisional Application No. 62/064,885, filed Oct. 16, 2014, which are hereby incorporated by reference herein.

### TECHNICAL FIELD

The technical field is related to bioeffective drugs and compositions for treating the body, and includes pharmaceutically acceptable implant systems comprising hydrogels that deliver a drug to an eye.

### BACKGROUND

Implants that deliver drugs over time in a therapeutically effective dosage are useful in many fields. The science of controlled drug release is diverse from a standpoint of both the range of scientific disciplines it encompasses and the range of its applications.

### SUMMARY

Hydrogels can be delivered at a choroid. The term at a choroid refers to suprachoroidal application and to application in the choroid. The hydrogels can be formed from hydrogel precursors that crosslink with each other in situ at the choroid. Further, syringes or the like equipped with biased or compressible members are taught herein for injections into an eye at a choroid.

### BRIEF DESCRIPTION OF THE FIGURES

FIG. 1 is a cross-sectional view of an eye, and shows a site of introduction at a choroid, specifically, at a suprachoroidal space.

FIG. 2 shows a hydrogel introduced at the site of FIG. 1, with an agent, at a choroid.

FIG. 3 shows release of the agent of FIG. 2 at a choroid, with the agent being preferentially released from the choroid side of the hydrogel implant. The hydrogel has cross-linked and stayed at the site of injection without flowing back into, or out of, the track of the needle at the site of injection.

FIG. 4 is a perspective view of a suprachoroidal space of the eye of FIG. 3, showing two examples, in panel (a) and panel (b), of a uniform and cohesive layer of the hydrogel formed at the choroid.

FIG. 5 illustrates injection at a choroid by application of force at a plunger of a syringe: at panel (a) the syringe is placed on the eye, at panel (b) the syringe is advanced into the sclera, at panel (c) the distal tip of the needle reaches a suprachoroidal position and expels its contents.

FIG. 6 illustrates injection at a choroid by application of force at a syringe having a constant reservoir pressure until delivery of its contents: at panel (a) the needle is on the sclera, at panel (b) the needle is advancing through the sclera, at panel (c) the distal tip of the needle exits the sclera.

FIG. 7 illustrates injection at a choroid with a syringe equipped with a biased member that exerts an increasing resistance as the needle penetrates the eye and indicates a force that is being applied at the needle: at rest at panel (a), a small force at panel (b) and a larger force at panel (c).

FIG. 8 depicts a syringe with a collapsible or biased member in-line with the needle.

FIG. 9 depicts a syringe with a movable needle in-line with a biased member at panel (a) and, and at panel (b), a syringe with a plunger that has a collapsible or biased member.

FIG. 10A depicts a syringe with a movable needle attached to a dashpot.

FIG. 10B depicts a two barreled syringe having a dashpot;

FIG. 10C depicts a two barreled syringe having a biasing member in one barrel;

FIG. 10D depicts a two barreled syringe having a biasing member in one barrel connected to a distal push rod;

FIG. 10E panel (a) depicts a needle inside a bias/damping member; at panel (b) the biasing member or damping member is compressed or engaged.

FIG. 11 depicts a needle with a dashpot and a needle with a restriction that increases resistance to movement of the plunger, with a dashpot being located proximal a midpoint of the barrel at panel (a) or distal a midpoint of the barrel at panel (b).

FIG. 12 illustrates delivery at a choroid that involves channeling or fluid dissection of part of a sclera.

FIG. 13 is an example of an in situ formed hydrogel as described in the Examples, showing delivery of an in situ formed hydrogel at a choroid (suprachoroidal). The sclera is dissected ad peeled back showing excellent adhesion to the sclera interior and a top of the choroid. The hydrogel formed a cohesive mass around the site of injection.

### DETAILED DESCRIPTION

Aqueous drug solutions and suspensions injected in the suprachoroidal space may be used to deliver a therapeutic agent (such as an active pharmaceutical ingredient (API)) at the choroid with the shortest path of diffusion from the suspension into the choroid and/or other surrounding tissues. The hydrogel can be formed so that, under certain conditions, drug clearance occurs closest to the hydrogel-tissue interface at the choroid. Hydrogel formulations can be tailored using factors such as: persistence of the hydrogel after implantation by controlling a hydrolysis-driven degradation rate; crosslinking conditions to facilitate control of the hydrogel distribution and shape as it crosslinks in situ following injection; and mechanical properties of the crosslinked material, such as modulus, hydrophilicity, strength, and cohesiveness.

Suspending the agent in a hydrogel can allow the agent to persist and be bioavailable for a longer duration in the suprachoroidal space than an aqueous solution or suspension. Longer persistence of therapeutic levels of the agent reduces the frequency that the agent needs to be readministered. A hydrogel reduces the agent washout that may occur with aqueous solutions and suspensions as well. Moreover, the agent can be disposed in various forms inside the hyrdrogel—in particles, in control-release particles (e.g., liposomes, micelles, capsules), as a solid, as an insoluble drug, and so forth. One such embodiment is to dispose the drug inside particles made of another hydrogel, with the various forms of the agent being options for disposition in one or both of the hydrogels. Further, a non-fluent hydrogel can help to seal an eye at the site of its introduction.

One of the challenges in this field is that the hydrogel should be a uniform material with a shape suited for delivery of the agent. A uniform material provides for consistent and controllable results. A single cohesive mass of material creates a three-dimensional space having a geometry suited to certain kinds of controlled release of the agent. Certain kinds of controlled release schemes are enabled when a cohesive mass is created. Alternatively, merely scattering particles containing an agent a short distance from their site of introduction at a choroid or other space of an eye provides a different delivery condition. Placement of the hydrogel between a choroid and the sclera presents enhanced opportunities for controlled release because the sclera is much less permeable to the diffusion of agents than the choroid. As a result, the sclera becomes part of the controlled release scheme and serves as a wall or layer for the implant. Experiments have indicated that one way to create a uniform mass at the choroid is to use a thick, viscous material. This material can be forced into the space and, when it is cohesive, it forms a uniform mass without being dissipated. Precursors that are cohesive at the pre-crosslinking stage may be chosen, as well as precursors that form cross-links with each other.

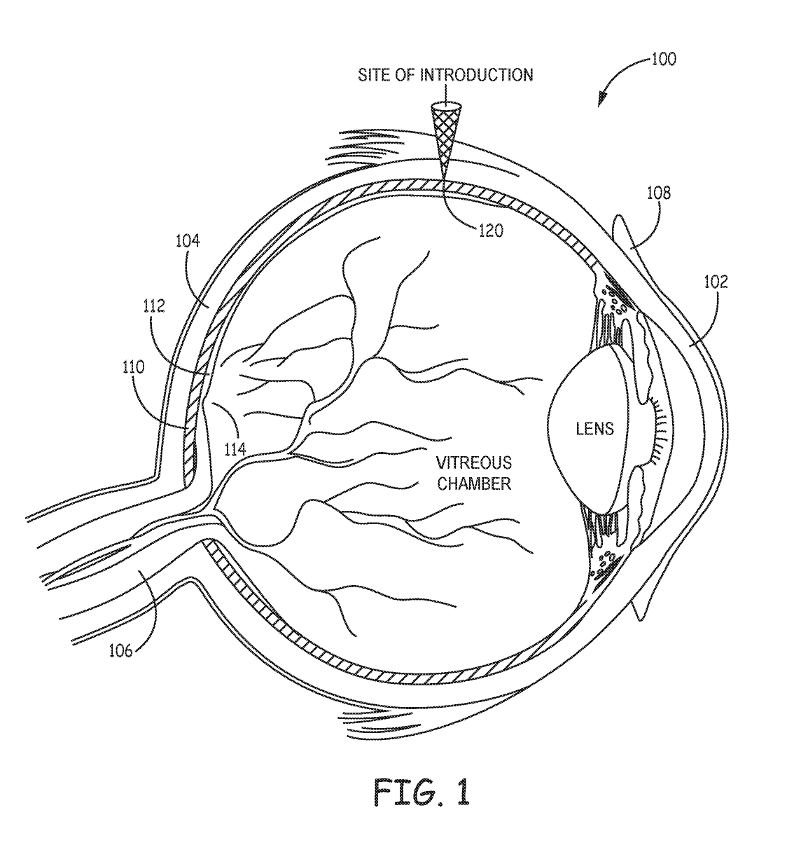

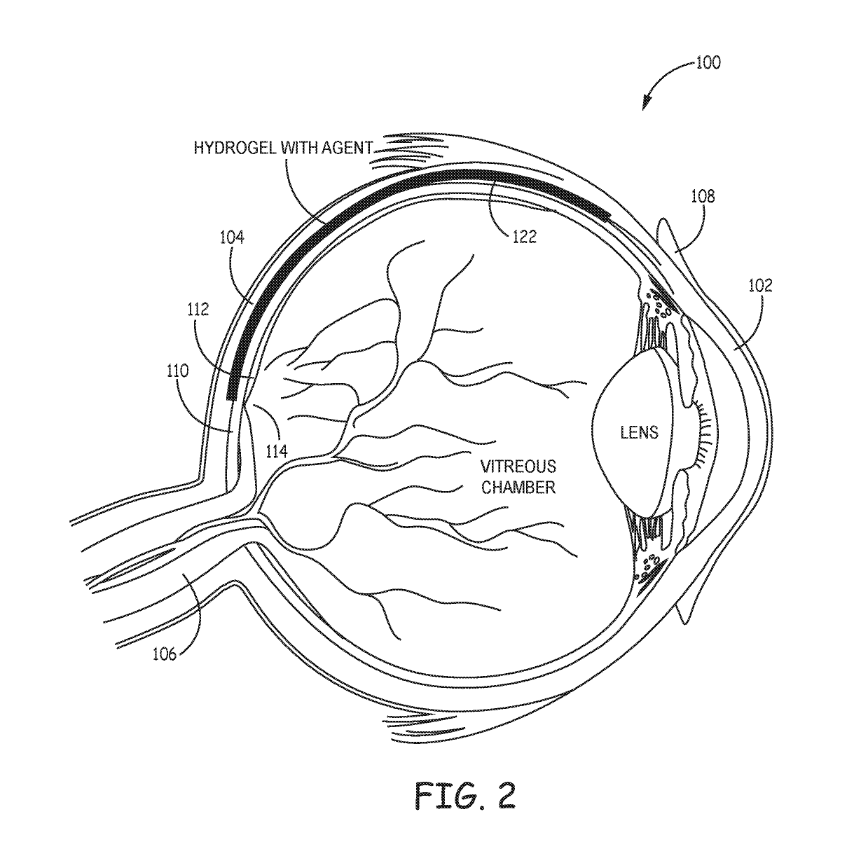

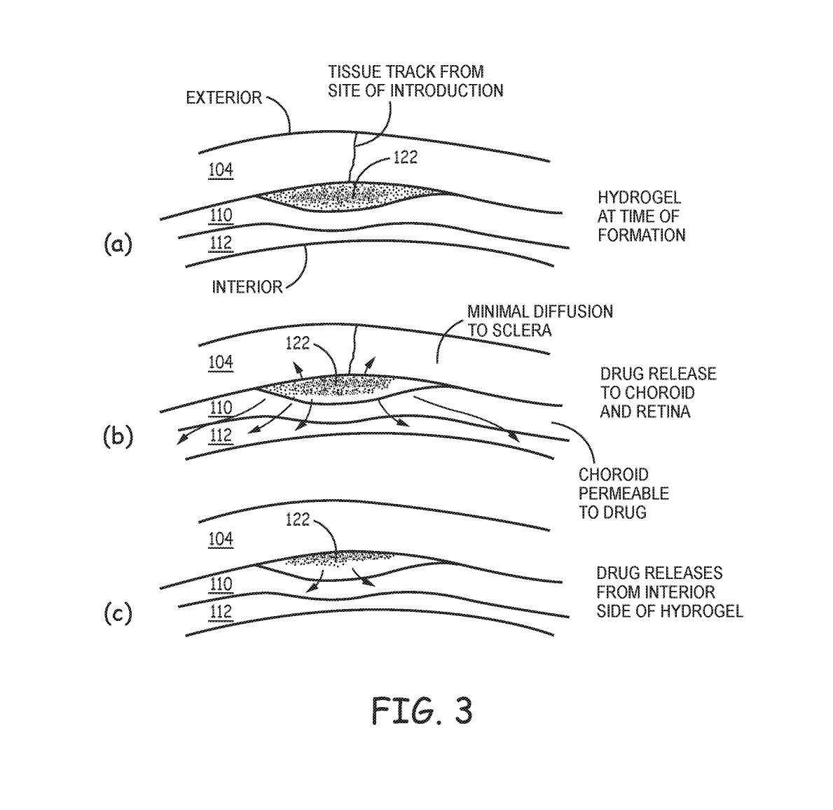

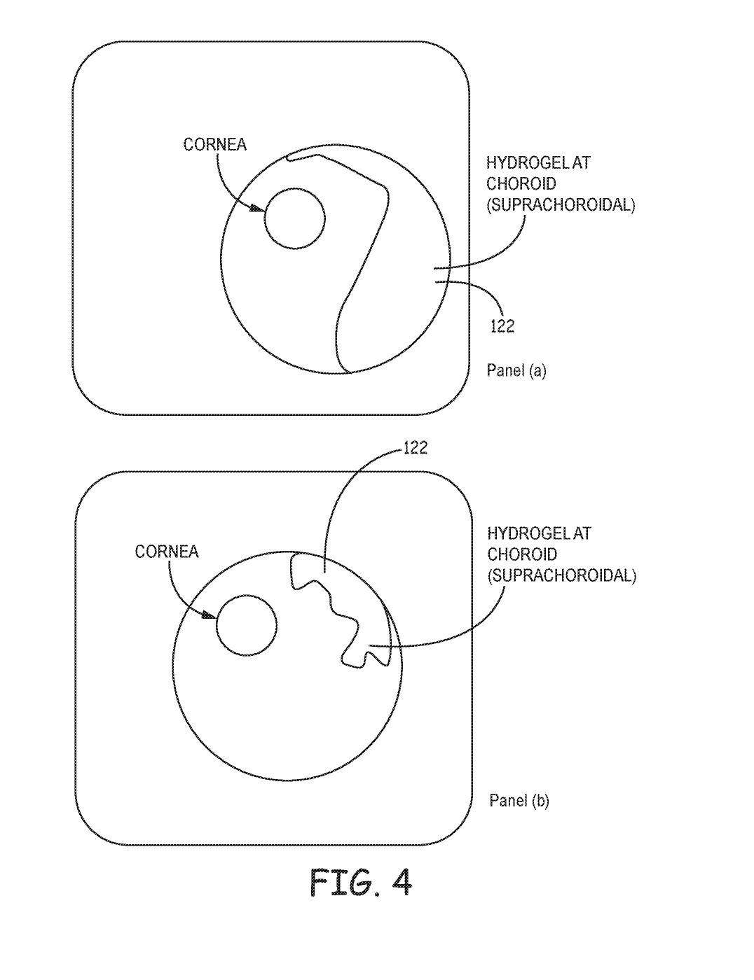

FIG. 1 depicts a site of injection 120 in eye 100. FIG. 2 shows cohesive hydrogel layer 122 at choroid 110. FIG. 3 depicts how placement next to the sclera 112 and at a suprachoroidal position, provides for a release from the layer 122 predominantly from its choroid (interior) side. FIG. 4 depicts how the layer, hydrogel 122, is spread at a choroid. Its composition is uniform, its shape is cohesive, and it is disposed as a layer having a height and an area. An area of contact with the choroid can be described as the area that is in a plane on top of the choroid; although the hydrogel might penetrate somewhat into a choroid, its area of contact with the choroid is useful for considering transport rates, distribution, and so forth. Accordingly, an area of contact could be, e.g., from 1% to 100% of the choroid area; artisans will immediately appreciate that all ranges and values within this range are contemplated and supported. An area of contact could be, e.g., from 0.5 to 60 mm²; artisans will immediately appreciate that all ranges and values within this range are contemplated and supported. A height, considered independently or in addition to an area of contact may be, e.g., 10 μm to 5 mm; Artisans will immediately appreciate that all ranges and values between the explicitly stated bounds are contemplated, with, e.g., any of the following being available as an upper or lower limit: 10, 20, 50, 100, 200, 500 microns, 1, 2, 3, 4, or 5 mm.

In general, to form a hydrogel, one or more precursors are reacted. The precursors form crosslinks that prevent the dissolution of the hydrogel in water. Precursors may be crosslinked via an ionic or covalent bond, a physical force, or other attraction. A covalent crosslink, however, will typically offer stability and predictability in reactant product architecture. To form covalently crosslinked hydrogels, the precursors are covalently crosslinked together. In general, precursors are joined to other precursors at two or more points, with each point being a linkage to the same or different polymers. Precursors with at least two reactive centers (for example, in free radical polymerization) can serve as crosslinkers since each reactive group can participate in the formation of a different growing polymer chain. In the case of functional groups without a reactive center, among others, crosslinking requires three or more such functional groups on at least one of the precursor types. For instance, many electrophilic-nucleophilic reactions consume the electrophilic and nucleophilic functional groups so that a third functional group is needed for the precursor to form a crosslink. Such precursors thus may have three or more functional groups and may be crosslinked by precursors with two or more functional groups. These are described in some detail, below.

### Devices

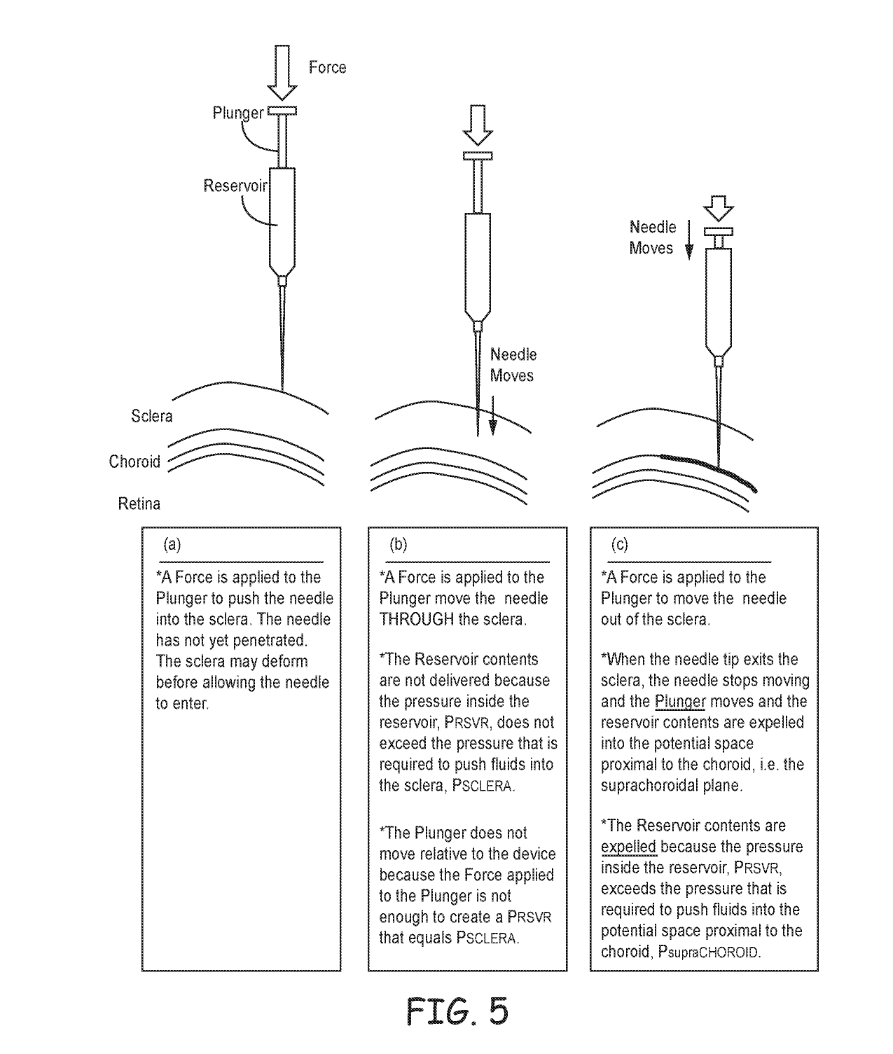

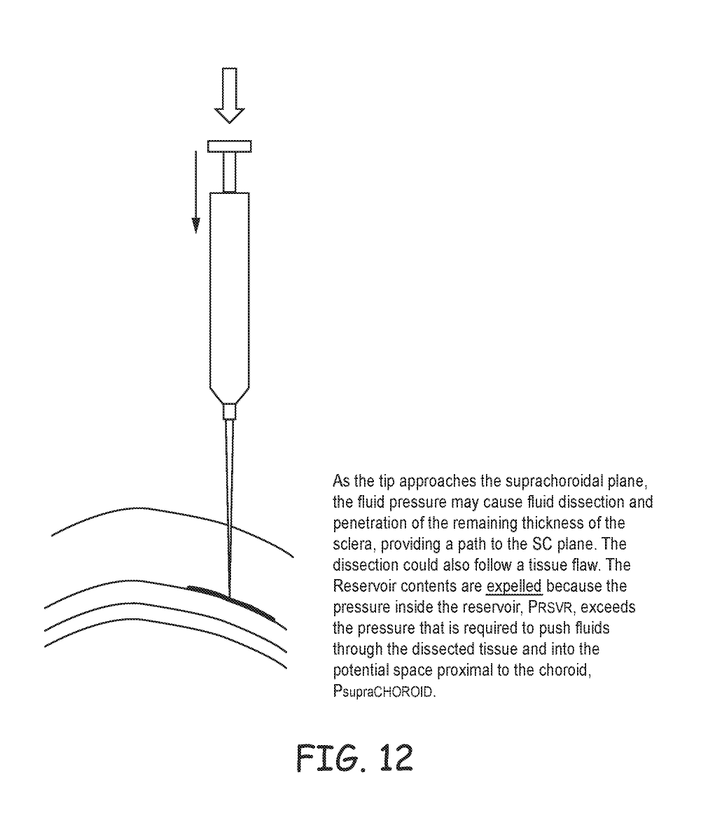

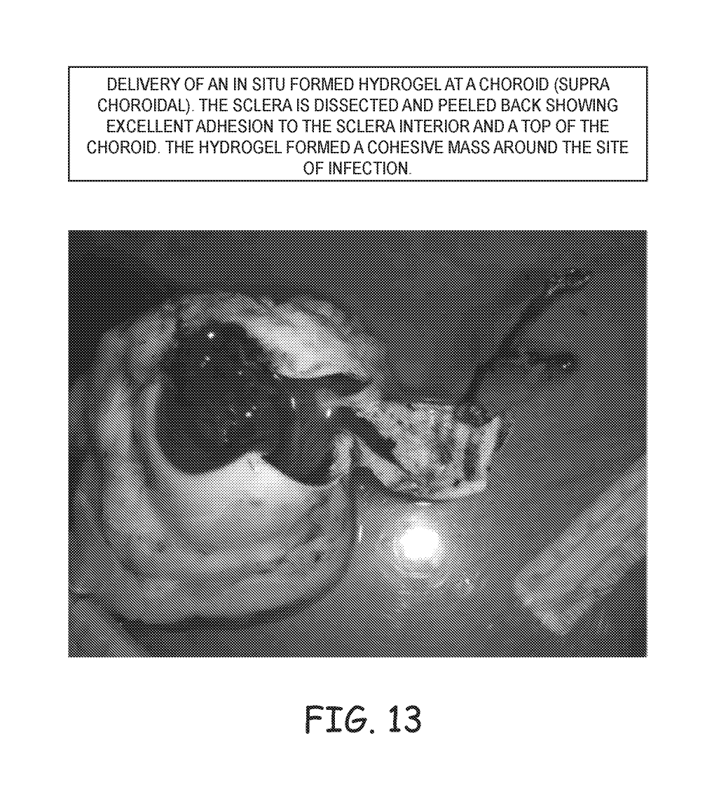

FIGS. 5-12 depict some embodiments of devices and methods for injecting materials at a choroid space or other location. Alternatively, conventional techniques for placement of a material at a choroid may be used. The materials, at the time of injection, may be fluent, meaning that they flow by themselves or in response to a force. Liquids are fluent materials, as are some gels.

Referring to FIG. 5, a syringe is prepared with a readily movable piston. The sclera is overlays the choroid, which overlays the retina. The sclera is thick compared to these other layers and a high pressure is required to force fluid into it: this pressure is PSCLERA. The pressure required to inject a fluid into a location above the choroid, PsupraCHOROID is significantly less than PSCLERA. The term syringe is broad and refers to sterile devices used to inject solutions or other fluent materials; these are typically devices having a piston or bulb or a comparably functioning member in fluid communication with a reservoir that is in fluid communication with an outlet, with the piston/bulb/member being actuated to expel the contents of the reservoir from the syringe. At FIG. 5 panel (a), a syringe needle is placed on the eye. When Force is applied to the plunger, the needle advances in the sclera, at panel (b), but the pressure inside the syringe (PRESERVOIR) and needle is less than PSCLERA. Once the needle is at the choroid as at FIG. 5 panel (c), however, PRESERVOIR is equal to or greater than PsupraCHOROID so that further force applied to the piston causes the piston to move and the contents of the reservoir to be expelled at the choroid. The surface area of the piston is chosen so that the Force (a product of the piston area and PRESERVOIR) is enough to move the needle in the sclera without exceeding PSCLERA. The piston is readily movable so that friction and pressure inside the syringe do not translate into a force that moves the needle farther into the eye. The needle may be chosen to increase or decrease an amount of force that is needed to move it through the sclera: for instance its gauge or a sharpness of the cutting edge of the needle may be increased or decreased and a bevel on its end may be adjusted, e.g., blunt, A, B, C, or custom bevel. In use, the syringe may comprise or be used in combination with a device to hold the piston at a desired angle, e.g., by using a hollow cylinder or other support that fits around the needle and/or syringe tube.

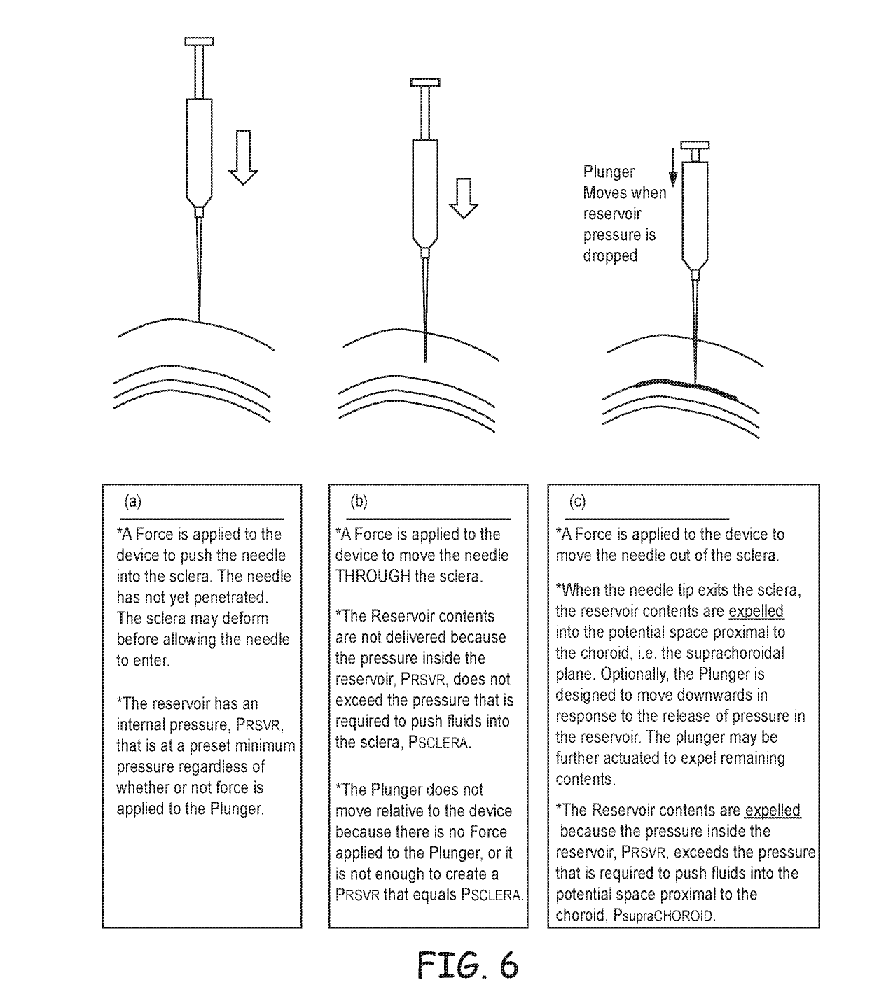

Referring to FIG. 6, at panel (a), a syringe is prepared with an internal pressure, PRESERVOIR that is less than PSCLERA and at least as much as PsupraCHOROID. The syringe needle is advanced, panel (b), through the sclera; at the choroid, at panel (c), the reservoir contents are expelled and the piston moves in response to a drop of pressure. A user then may push the rest of the contents out of the syringe by further movement of the piston, e.g., by pushing the plunger.

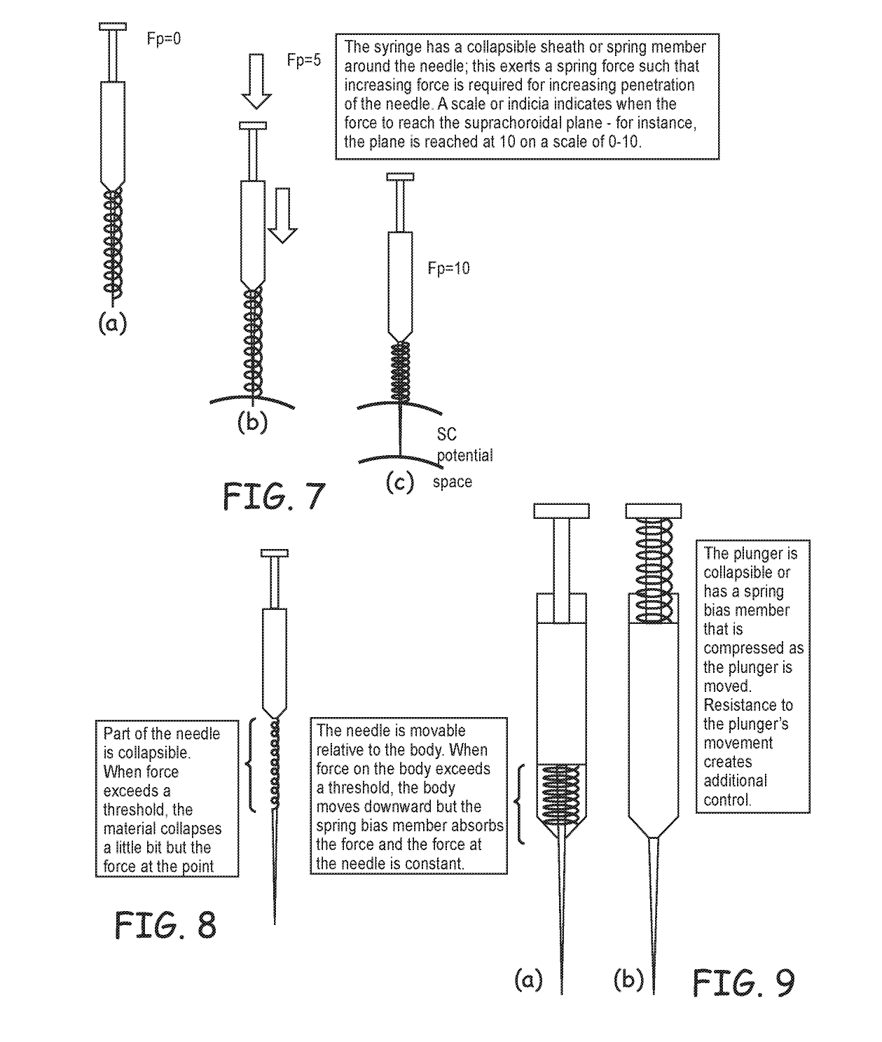

FIG. 7 depicts a syringe with a collapsible sheath or spring member. A predetermined force is required to move the needle to the choroid. The force increases as the needle approaches the choroid. A scale may be provided with the device that provides an indication of the force or depth of penetration into the eye.

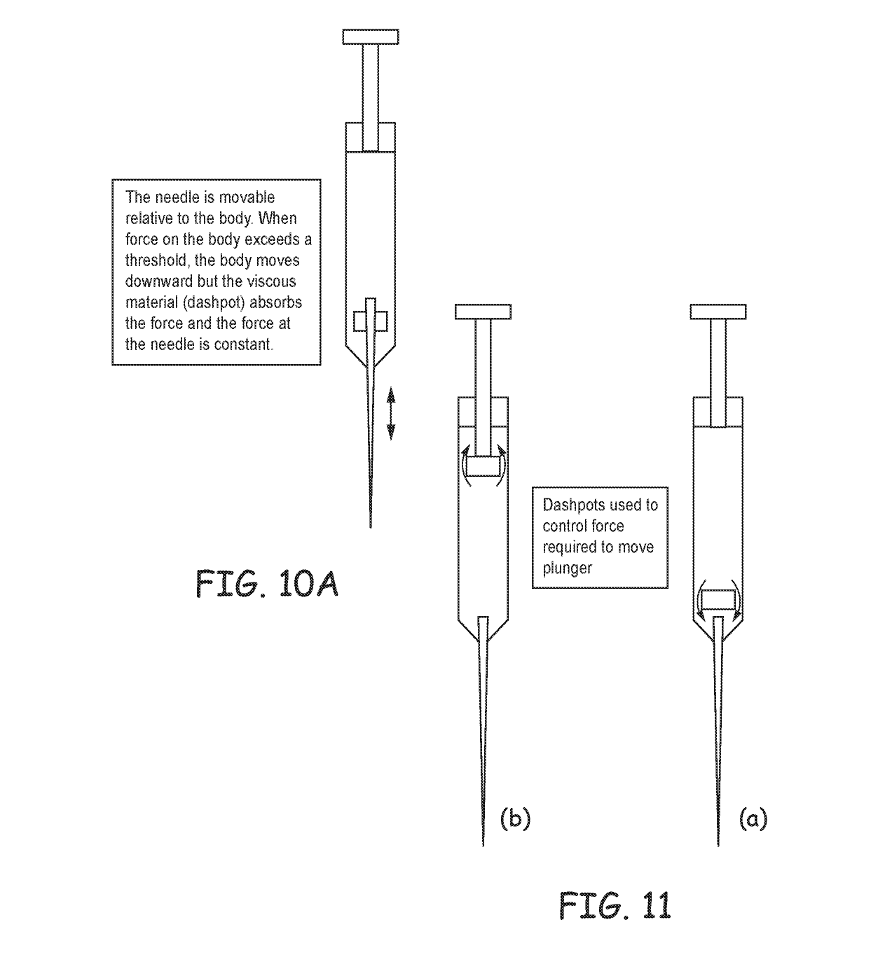

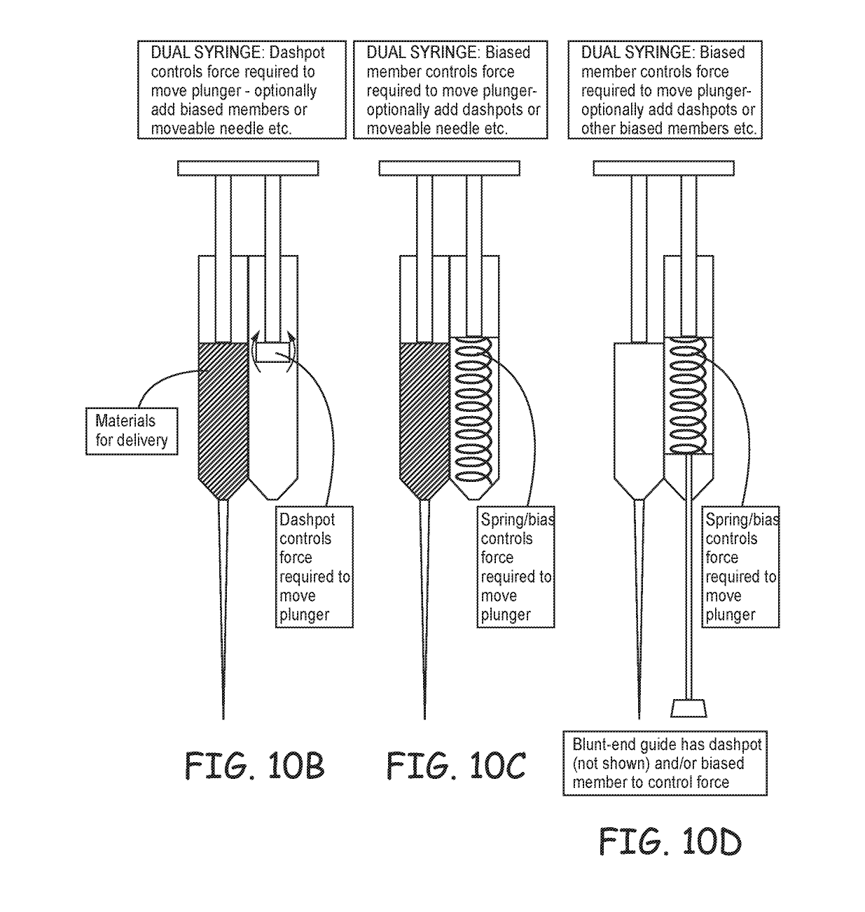

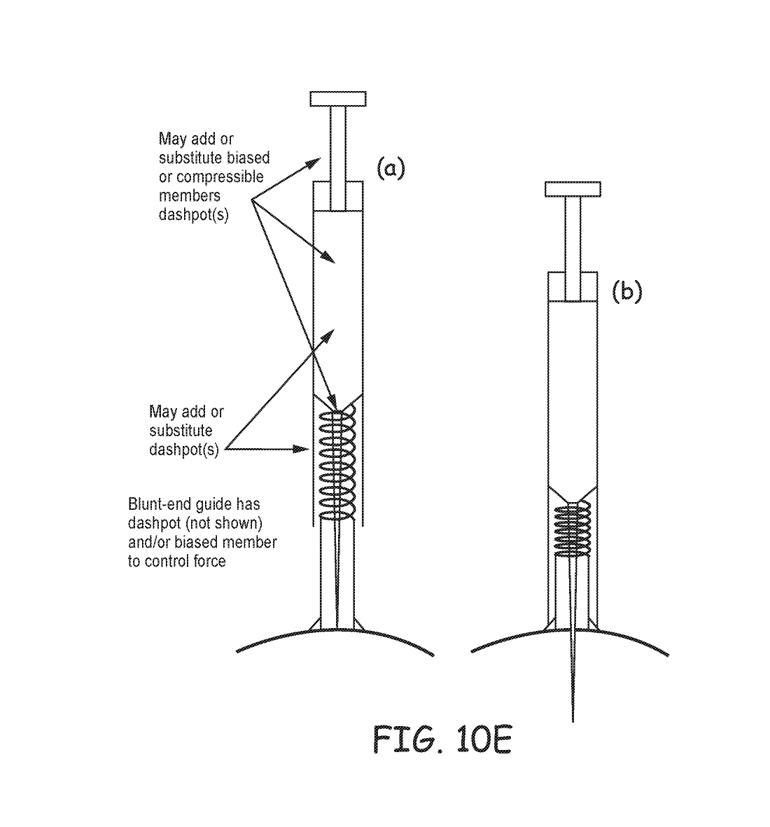

FIGS. 8-9 illustrate a movable needle or plunger. A user applies force to the syringe and/or plunger and a collapsible material or a biased member is compressed to absorb the force. The material/biased member may be adjusted so that force within a reasonable expected range is translated into a constant force at the needle. FIGS. 10-11 depicts dashpots or the equivalent for control of a force applied at the needle tip. The sheaths/materials/biased members/dashpots/restricted flow areas may be used independently or in combination.

The sclera may, in some cases, break down before a needle tip is at the choroid: the pressure in the syringe is less than PSCLERA but the sclera has less support and strength near its edge, or there may be a scleral flaw or irregularity. As a result, the contents of the syringe may channel through the sclera to position at the choroid, with the hydrogel being effectively formed at the choroid.

Syringes, needles, and the like are well known and may be provided in a wide range of materials, including various glasses or plastics, and as disposable, reusable, or sterile devices. Similarly, biasing members, e.g., springs, dashpots, permanently or reversibly compressible materials, spring bias members (a term that includes a spring or other members to provide a bias force), and the like are known to artisans, who will be able to use them to make the delivery devices after reading this disclosure.

### Precursor Materials

The hydrogels are made from precursors. Precursors are chosen in consideration of the properties that are desired for the resultant hydrogel. There are various suitable precursors for use in making the hydrogels. The term precursor refers to those molecules crosslinked to form the hydrogel or organogel matrix. While other materials might be present in the hydrogel or organogel, such as therapeutic agents or fillers, they are not precursors. Hydrogels are materials that do not dissolve in water and retain a significant fraction (more than 20%) of water within their structure. In fact, water contents in excess of 90% are often known. Hydrogels may be formed by crosslinking water soluble molecules to form networks of essentially infinite molecular weight. Hydrogels with high water contents are typically soft, pliable materials. Hydrogels and drug delivery systems as described in U.S. Publication Nos. 2009/0017097, 2011/0142936 and 2012/0071865 may be adapted for use with the materials and methods herein by following the guidance provided herein; these references are hereby incorporated herein by reference for all purposes, and in case of conflict, the instant specification is controlling.

Hydrogels or fluent gels or other fluent materials may be formed from natural, synthetic, or biosynthetic polymers. Natural polymers may include glycosminoglycans, polysaccharides, and proteins. Some examples of glycosaminoglycans include dermatan sulfate, hyaluronic acid, the chondroitin sulfates, chitin, heparin, keratan sulfate, keratosulfate, and derivatives thereof. In general, the glycosaminoglycans are extracted from a natural source and purified and derivatized. However, they also may be synthetically produced or synthesized by modified microorganisms such as bacteria. These materials may be modified synthetically from a naturally soluble state to a partially soluble or water swellable or hydrogel state. This modification may be accomplished by various well-known techniques, such as by conjugation or replacement of ionizable or hydrogen bondable functional groups such as carboxyl and/or hydroxyl or amine groups with other more hydrophobic groups.

For example, carboxyl groups on hyaluronic acid may be esterified by alcohols to decrease the solubility of the hyaluronic acid. Such processes are used by various manufacturers of hyaluronic acid products (such as Genzyme Corp., Cambridge, Mass.) to create hyaluronic acid based sheets, fibers, and fabrics that form hydrogels. Other natural polysaccharides, such as carboxymethyl cellulose or oxidized regenerated cellulose, natural gum, agar, agrose, sodium alginate, carrageenan, fucoidan, furcellaran, laminaran, hypnea, eucheuma, gum arabic, gum ghatti, gum karaya, gum tragacanth, locust beam gum, arbinoglactan, pectin, amylopectin, gelatin, hydrophilic colloids such as carboxymethyl cellulose gum or alginate gum crosslinked with a polyol such as propylene glycol, and the like, also form hydrogels upon contact with aqueous surroundings.

Synthetic gels or hydrogels may be biostable or biodegradable. Examples of biostable hydrophilic polymeric materials are poly(hydroxyalkyl methacrylate), poly(electrolyte complexes), poly(vinylacetate) cross-linked with hydrolysable or otherwise degradable bonds, and water-swellable N-vinyl lactams. Other hydrogels include hydrophilic hydrogels known as CARBOPOL®, an acidic carboxy polymer (Carbomer resins are high molecular weight, allylpentaerythritol-crosslinked, acrylic acid-based polymers, modified with C10-C30 alkyl acrylates), polyacrylamides, polyacrylic acid, starch graft copolymers, acrylate polymer, ester cross-linked polyglucan. Such hydrogels are described, for example, in U.S. Pat. No. 3,640,741 to Etes, U.S. Pat. No. 3,865,108 to Hartop, U.S. Pat. No. 3,992,562 to Denzinger et al., U.S. Pat. No. 4,002,173 to Manning et al., U.S. Pat. No. 4,014,335 to Arnold and U.S. Pat. No. 4,207,893 to Michaels, all of which are incorporated herein by reference, with the present specification controlling in case of conflict.

Hydrogels may be made from precursors. The precursors are crosslinked with each other. Crosslinks can be formed by covalent bonds or physical bonds. Examples of physical bonds are ionic bonds, hydrophobic association of precursor molecule segments, and crystallization of precursor molecule segments. The precursors can be triggered to react to form a crosslinked hydrogel. The precursors can be polymerizable and include crosslinkers that are often, but not always, polymerizable precursors. Polymerizable precursors are thus precursors that have functional groups that react with each other to form matrices and/or polymers made of repeating units. Precursors may be polymers.

Some precursors thus react by chain-growth polymerization, also referred to as addition polymerization, and involve the linking together of monomers incorporating double or triple chemical bonds. These unsaturated monomers have extra internal bonds which are able to break and link up with other monomers to form the repeating chain. Monomers are polymerizable molecules with at least one group that reacts with other groups to form a polymer. A macromonomer (or macromer) is a polymer or oligomer that has at least one reactive group, often at the end, which enables it to act as a monomer; each macromonomer molecule is attached to the polymer by reaction the reactive group. Thus macromonomers with two or more monomers or other functional groups tend to form covalent crosslinks. Addition polymerization is involved in the manufacture of, e.g., polypropylene or polyvinyl chloride. One type of addition polymerization is living polymerization.

Some precursors thus react by condensation polymerization that occurs when monomers bond together through condensation reactions. Typically these reactions can be achieved through reacting molecules incorporating alcohol, amine or carboxylic acid (or other carboxyl derivative) functional groups. When an amine reacts with a carboxylic acid an amide or peptide bond is formed, with the release of water. Some condensation reactions follow a nucleophilic acyl substitution, e.g., as in U.S. Pat. No. 6,958,212, which is hereby incorporated by reference herein in its entirety to the extent it does not contradict what is explicitly disclosed herein. Some precursors react by a chain growth mechanism. Chain growth polymers are defined as polymers formed by the reaction of monomers or macromonomers with a reactive center. A reactive center is a particular location within a chemical compound that is the initiator of a reaction in which the chemical is involved. In chain-growth polymer chemistry, this is also the point of propagation for a growing chain. The reactive center is commonly radical, anionic, or cationic in nature, but can also take other forms. Chain growth systems include free radical polymerization, which involves a process of initiation, propagation and termination. Initiation is the creation of free radicals necessary for propagation, as created from radical initiators, e.g., organic peroxide molecules. Termination occurs when a radical reacts in a way that prevents further propagation. The most common method of termination is by coupling where two radical species react with each other forming a single molecule. Some precursors react by a step growth mechanism, and are polymers formed by the stepwise reaction between functional groups of monomers. Most step growth polymers are also classified as condensation polymers, but not all step growth polymers release condensates. Monomers may be polymers or small molecules. A polymer is a high molecular weight molecule formed by combining many smaller molecules (monomers) in a regular pattern. Oligomers are polymers having less than about 20 monomeric repeat units. A small molecule generally refers to a molecule that is less than about 2000 Daltons. The precursors may thus be small molecules, such as acrylic acid or vinyl caprolactam, larger molecules containing polymerizable groups, such as acrylate-capped polyethylene glycol (PEG-diacrylate), or other polymers containing ethylenically-unsaturated groups, such as those of U.S. Pat. No. 4,938,763 to Dunn et al, U.S. Pat. Nos. 5,100,992 and 4,826,945 to Cohn et al, or U.S. Pat. Nos. 4,741,872 and 5,160,745 to DeLuca et al., each of which is hereby incorporated by reference herein in its entirety to the extent it does not contradict what is explicitly disclosed herein.

In some embodiments, each precursor is multifunctional, meaning that it comprises two or more electrophilic or nucleophilic functional groups, such that a nucleophilic functional group on one precursor may react with an electrophilic functional group on another precursor to form a covalent bond. At least one of the precursors comprises more than two functional groups, so that, as a result of electrophilic-nucleophilic reactions, the precursors combine to form crosslinked polymeric products.

The precursors may have biologically inert and hydrophilic portions, e.g., a core. In the case of a branched polymer, a core refers to a contiguous portion of a molecule joined to arms that extend from the core, with the arms having a functional group, which is often at the terminus of the branch. A hydrophilic molecule, e.g., a precursor or precursor portion, has a solubility of at least 1 g/100 mL in an aqueous solution. A hydrophilic portion may be, for instance, a polyether, for example, polyalkylene oxides such as polyethylene glycol (PEG), polyethylene oxide (PEO), polyethylene oxide-co-polypropylene oxide (PPO), co-polyethylene oxide block or random copolymers, and polyvinyl alcohol (PVA), poly (vinyl pyrrolidinone) (PVP), poly (amino acids, dextran, or a protein. The precursors may have a polyalkylene glycol portion and may be polyethylene glycol based, with at least about 80% or 90% by weight of the polymer comprising polyethylene oxide repeats. The polyethers and more particularly poly (oxyalkylenes) or poly (ethylene glycol) or polyethylene glycol are generally hydrophilic. As is customary in these arts, the term PEG is used to refer to PEO with or without hydroxyl end groups.

A precursor may also be a macromolecule (or macromer), which is a molecule having a molecular weight in the range of a thousand to many millions. The hydrogel or organogel however, may be made with at least one of the precursors as a small molecule of about 1000 Da or less (alternatively: 2000 Da or less). The macromolecule, when reacted in combination with a small molecule (of about 1000 Da or less/200 Da or less), is preferably at least five to fifty times greater in molecular weight than the small molecule and is preferably less than about 60,000 Da; artisans will immediately appreciate that all the ranges and values within the explicitly stated ranges are contemplated. A more preferred range is a macromolecule that is about seven to about thirty times greater in molecular weight than the crosslinker and a most preferred range is about ten to twenty times difference in weight. Further, a macromolecular molecular weight of 5,000 to 50,000 is useful, as is a molecular weight of 7,000 to 40,000 or a molecular weight of 10,000 to 20,000. There are certain advantage to having a small molecule, such as diffusivity for completion of reactions.

Certain macromeric precursors are the crosslinkable, biodegradable, water-soluble macromers described in U.S. Pat. No. 5,410,016 to Hubbell et al, which is hereby incorporated herein by reference in its entirety to the extent it does not contradict what is explicitly disclosed. These macromers are characterized by having at least two polymerizable groups, separated by at least one degradable region.

Synthetic precursors may be used. Synthetic refers to a molecule not found in nature or not normally found in a human. Some synthetic precursors are free of amino acids or free of amino acid sequences that occur in nature. Some synthetic precursors are polypeptides that are not found in nature or are not normally found in a human body, e.g., di-, tri-, or tetra-lysine. Some synthetic molecules have amino acid residues but only have one, two, or three that are contiguous, with the amino acids or clusters thereof being separated by non-natural polymers or groups. Polysaccharides or their derivatives are thus not synthetic.

Alternatively, natural proteins or polysaccharides may be adapted for use as gels, hydrogels, or other materials for use with these methods, e.g., collagens, fibrin(ogen)s, albumins, alginates, hyaluronic acid, and heparins. These natural molecules may further include chemical derivitization, e.g., synthetic polymer decorations. The natural molecule may be crosslinked via its native nucleophiles or after it is derivatized with functional groups, e.g., as in U.S. Pat. Nos. 5,304,595, 5,324,775, 6,371,975, and 7,129,210, each of which is hereby incorporated by reference to the extent it does not contradict what is explicitly disclosed herein. Natural refers to a molecule found in nature. Natural polymers, for example proteins or glycosaminoglycans, e.g., collagen, fibrinogen, albumin, and fibrin, may be crosslinked using reactive precursor species with electrophilic functional groups. Natural polymers normally found in the body are proteolytically degraded by proteases present in the body. Such polymers may be reacted via functional groups such as amines, thiols, or carboxyls on their amino acids or derivatized to have activatable functional groups. While natural polymers may be used in hydrogels, their time to gelation and ultimate mechanical properties must be controlled by appropriate introduction of additional functional groups and selection of suitable reaction conditions, e.g., pH.

Precursors may be made with a hydrophobic portion provided that the resultant hydrogel retains the requisite amount of water, e.g., at least about 20%. In some cases, the precursor is nonetheless soluble in water because it also has a hydrophilic portion. In other cases, the precursor makes dispersion in the water (a suspension) but is nonetheless reactable to from a crosslinked material. Some hydrophobic portions may include a plurality of alkyls, polypropylenes, alkyl chains, or other groups. Some precursors with hydrophobic portions are sold under the trade names PLURONIC F68, JEFFAMINE, or TECTRONIC. A hydrophobic molecule or a hydrophobic portion of a copolymer or the like is one that is sufficiently hydrophobic to cause the molecule (e.g., polymer or copolymer) to aggregate to form micelles or microphases involving the hydrophobic domains in an aqueous continuous phase or one that, when tested by itself, is sufficiently hydrophobic to precipitate from, or otherwise change phase while within, an aqueous solution of water at pH from about 7 to about 7.5 at temperatures from about 30 to about 50 degrees Centigrade.

Precursors may have, e.g., 2-100 arms, with each arm having a terminus, bearing in mind that some precursors may be dendrimers or other highly branched materials. An arm on a hydrogel precursor refers to a linear chain of chemical groups that connect a crosslinkable functional group to a polymer core. Some embodiments are precursors with between 3 and 300 arms; artisans will immediately appreciate that all the ranges and values within the explicitly stated ranges are contemplated, e.g., 4 to 16, 8 to 100, or at least 6 arms.

Thus hydrogels can be made, e.g., from a multi-armed precursor with a first set of functional groups and a low molecular-weight precursor having a second set of functional groups. For example, a six-armed or eight-armed precursor may have hydrophilic arms, e.g., polyethylene glycol, terminated with primary amines, with the molecular weight of the arms being about 1,000 to about 40,000; artisans will immediately appreciate that all ranges and values within the explicitly stated bounds are contemplated. Such precursors may be mixed with relatively smaller precursors, for example, molecules with a molecular weight of between about 100 and about 5000, or no more than about 800, 1000, 2000, or 5000 having at least about three functional groups, or between about 3 to about 16 functional groups; ordinary artisans will appreciate that all ranges and values between these explicitly articulated values are contemplated. Such small molecules may be polymers or non-polymers and natural or synthetic.

Precursors that are not dendrimers may be used. Dendritic molecules are highly branched radially symmetrical polymers in which the atoms are arranged in many arms and subarms radiating out from a central core. Dendrimers are characterized by their degree of structural perfection as based on the evaluation of both symmetry and polydispersity and require particular chemical processes to synthesize. Accordingly, an artisan can readily distinguish dendrimer precursors from non-dendrimer precursors. Dendrimers have a shape that is typically dependent on the solubility of its component polymers in a given environment, and can change substantially according to the solvent or solutes around it, e.g., changes in temperature, pH, or ion content.

Precursors may be dendrimers, e.g., as in U.S. Publication Nos. 2004/0086479 and 2004/0131582 and PCT Publication Nos. WO07005249, WO07001926 and WO06031358, or the U.S. counterparts thereof; dendrimers may also be useful as multifunctional precursors, e.g., as in U.S. Publication Nos. 2004/0131582 and 2004/0086479 and PCT Publication Nos. WO06031388 and WO06031388; each of which US and PCT applications are hereby incorporated by reference herein in its entirety to the extent they do not contradict what is explicitly disclosed herein. Dendrimers are highly ordered possess high surface area to volume ratios, and exhibit numerous end groups for potential functionalization. Embodiments include multifunctional precursors that are not dendrimers.

Some embodiments include a precursor that consists essentially of an oligopeptide sequence of no more than five residues, e.g., amino acids comprising at least one amine, thiol, carboxyl, or hydroxyl side chain. A residue is an amino acid, either as occurring in nature or derivatized thereof. The backbone of such an oligopeptide may be natural or synthetic. In some embodiments, peptides of two or more amino acids are combined with a synthetic backbone to make a precursor; certain embodiments of such precursors have a molecular weight in the range of about 100 to about 10,000 or about 300 to about 500 Artisans will immediately appreciate that all ranges and values between these explicitly articulated bounds are contemplated.

Precursors may be prepared to be free of amino acid sequences cleavable by enzymes present at the site of introduction, including free of sequences susceptible to attach by metalloproteinases and/or collagenases. Further, precursors may be made to be free of all amino acids, or free of amino acid sequences of more than about 50, 30, 20, 10, 9, 8, 7, 6, 5, 4, 3, 2, or 1 amino acids. Precursors may be non-proteins, meaning that they are not a naturally occurring protein and cannot be made by cleaving a naturally occurring protein and cannot be made by adding synthetic materials to a protein. Precursors may be non-collagen, non-fibrin, non-fibrinogen, and non-albumin, meaning that they are not one of these proteins and are not chemical derivatives of one of these proteins. The use of non-protein precursors and limited use of amino acid sequences can be helpful for avoiding immune reactions, avoiding unwanted cell recognition, and avoiding the hazards associated with using proteins derived from natural sources. Precursors can also be non-saccharides (free of saccharides) or essentially non-saccharides (free of more than about 5% saccharides by w/w of the precursor molecular weight. Thus a precursor may, for example, exclude hyaluronic acid, heparin, or gellan. Precursors can also be both non-proteins and non-saccharides.

Peptides may be used as precursors. In general, peptides with less than about 10 residues are preferred, although larger sequences (e.g., proteins) may be used. Artisans will immediately appreciate that every range and value within these explicit bounds is included, e.g., 1-10, 2-9, 3-10, 1, 2, 3, 4, 5, 6, or 7. Some amino acids have nucleophilic groups (e.g., primary amines or thiols) or groups that can be derivatized as needed to incorporate nucleophilic groups or electrophilic groups (e.g., carboxyls or hydroxyls). Polyamino acid polymers generated synthetically are normally considered to be synthetic if they are not found in nature and are engineered not to be identical to naturally occurring biomolecules.

Some gels or hydrogels are made with a polyethylene glycol-containing precursor. Polyethylene glycol (PEG, also referred to as polyethylene oxide when occurring in a high molecular weight) refers to a polymer with a repeat group (CH₂CH₂O)ₙ, with n being at least 3. A polymeric precursor having a polyethylene glycol thus has at least three of these repeat groups connected to each other in a linear series. The polyethylene glycol content of a polymer or arm is calculated by adding up all of the polyethylene glycol groups on the polymer or arm, even if they are interrupted by other groups. Thus, an arm having at least 1000 MW polyethylene glycol has enough CH₂CH₂O groups to total at least 1000 MW. As is customary terminology in these arts, a polyethylene glycol polymer does not necessarily refer to a molecule that terminates in a hydroxyl group. Molecular weights are abbreviated in thousands using the symbol k, e.g., with 15 K meaning 15,000 molecular weight, i.e., 15,000 Daltons. NH2 refers to an amine termination. SG refers to succinimidyl glutarate. SS refers to succinimidyl succinate. SAP refers to succinimidyl adipate. SAZ refers to succinimidyl azelate. SS, SG, SAP and SAZ are succinimidyl esters that have an ester group that degrades by hydrolysis in water. Hydrolytically degradable or water-degradable thus refers to a material that would spontaneously degrade in vitro in an excess of water without any enzymes or cells present to mediate the degradation. A time for degradation refers to effective disappearance of the material as judged by the naked eye. Trilysine (also abbreviated LLL) is a synthetic tripeptide. PEG and/or hydrogels, as well as compositions that comprise the same, may be provided in a form that is pharmaceutically acceptable, meaning that it is highly purified and free of contaminants, e.g., pyrogens.

### Material Structures

A gel's or hydrogel's structure and its constituent material composition, e.g., of the hydrogel's precursors determine its properties. Precursor factors include properties such as biocompatibility, water solubility, hydrophilicity, molecular weight, arm length, number of arms, functional groups, distance between crosslinks, degradability, and the like. The choice of reaction conditions also effects the hydrogel's structure and properties, including choices of solvents, reaction schemes, reactant concentrations, solids content, and the like. There can be a variety of ways to achieve certain properties, or combination of properties. On the other hand some properties are in tension with each other, for instance brittleness may increase as a distance between crosslinks decreases or solids content increases. Strength may be increased by increasing the number of crosslinks but swelling may thereby be reduced. Artisans will appreciate that the same materials may be used to make matrices with a great range of structures that will have highly distinct mechanical properties and performance, such that the achievement of a particular property should not be merely assumed based on the general types of precursors that are involved.

The spacing between molecular strands of the hydrogel (the matrix) affects several hydrogel properties, including a rate of diffusion of molecules. The crosslinking density can be controlled by the choice of the overall molecular weight of the precursor(s) used as crosslinker(s) and other precursor(s) and the number of functional groups available per precursor molecule. A lower molecular weight between crosslinks such as 200 will give much higher crosslinking density as compared to a higher molecular weight between crosslinks such as 500,000; artisans will immediately appreciate that all ranges and values within this range are contemplated and supported, e.g., 200 to 250,000, 500 to 400,000, and so forth. The crosslinking density also may be controlled by the overall percent solids of the crosslinker and functional polymer solutions. Yet another method to control crosslink density is by adjusting the stoichiometry of nucleophilic functional groups to electrophilic functional groups. A one to one ratio leads to the highest crosslink density. Precursors with longer distances between crosslinkable sites form gels that are generally softer, more compliant, and more elastic. Thus an increased length of a water-soluble segment, such as a polyethylene glycol, tends to enhance elasticity to produce desirable physical properties. Thus certain embodiments are directed to precursors with water soluble segments having molecular weights in the range of 1,000 to 100,000; artisans will immediately appreciate that all the ranges and values within the explicitly stated ranges are contemplated, e.g. 5,000 to 35,000. The solids content of the hydrogel can affect its mechanical properties and biocompatibility and reflects a balance between competing requirements. A relatively low solids content is useful, e.g., between about 2.5% to about 20%, including all ranges and values there between, e.g., about 2.5% to about 10%, about 5% to about 15%, or less than about 15%.

### Functional Groups

The precursors for covalent crosslinking have functional groups that react with each other to form the material via covalent bonds, either outside a patient, or in situ. The functional groups generally are polymerizable, a broad category that encompasses free radical, addition, and condensation polymerization and also groups for electrophile-nucleophile reactions. Various aspects of polymerization reactions are discussed in the precursors section herein.

Thus in some embodiments, precursors have a polymerizable group that is activated by photoinitiation or redox systems as used in the polymerization arts, or electrophilic functional groups, for instance: carbodiimidazole, sulfonyl chloride, chlorocarbonates, n-hydroxysuccinimidyl ester, succinimidyl ester or sulfasuccinimidyl esters, or as in U.S. Pat. No. 5,410,016 or 6,149,931, each of which are hereby incorporated by reference herein in its entirety to the extent they do not contradict what is explicitly disclosed herein. The nucleophilic functional groups may be, for example, amine, hydroxyl, carboxyl, and thiol. Another class of electrophiles are acyls, e.g., as in U.S. Pat. No. 6,958,212, which describes, among other things, Michael addition schemes for reacting polymers.

Certain functional groups, such as alcohols or carboxylic acids, do not normally react with other functional groups, such as amines, under physiological conditions (e.g., pH 7.2-11.0, 37° C.). However, such functional groups can be made more reactive by using an activating group such as N-hydroxysuccinimide. Certain activating groups include carbonyldiimidazole, sulfonyl chloride, aryl halides, sulfosuccinimidyl esters, N-hydroxysuccinimidyl ester, succinimidyl ester, epoxide, aldehyde, maleimides, imidoesters and the like. The N-hydroxysuccinimide esters or N-hydroxysulfosuccinimide (NHS) groups are useful groups for crosslinking of proteins or amine-containing polymers, e.g., amino terminated polyethylene glycol. An advantage of an NHS-amine reaction is that the reaction kinetics are favorable, but the gelation rate may be adjusted through pH or concentration. The NHS-amine crosslinking reaction leads to formation of N-hydroxysuccinimide as a side product. Sulfonated or ethoxylated forms of N-hydroxysuccinimide have a relatively increased solubility in water and hence their rapid clearance from the body. An NHS-amine crosslinking reaction may be carried out in aqueous solutions and in the presence of buffers, e.g., phosphate buffer (pH 5.0-7.5), triethanolamine buffer (pH 7.5-9.0), or borate buffer (pH 9.0-12), or sodium bicarbonate buffer (pH 9.0-10.0). Aqueous solutions of NHS based crosslinkers and functional polymers preferably are made just before the crosslinking reaction due to reaction of NHS groups with water. The reaction rate of these groups may be delayed by keeping these solutions at lower pH (pH 4-7). Buffers may also be included in the hydrogels introduced into a body.

In some embodiments, each precursor comprises only nucleophilic or only electrophilic functional groups, so long as both nucleophilic and electrophilic precursors are used in the crosslinking reaction. Thus, for example, if a crosslinker has nucleophilic functional groups such as amines, the functional polymer may have electrophilic functional groups such as N-hydroxysuccinimides. On the other hand, if a crosslinker has electrophilic functional groups such as sulfosuccinimides, then the functional polymer may have nucleophilic functional groups such as amines or thiols. Thus, functional polymers such as proteins, poly(allyl amine), or amine-terminated di- or multifunctional poly(ethylene glycol) can be used.

One embodiment has reactive precursor species with 2 to 16 nucleophilic functional groups each and reactive precursor species with 2 to 16 electrophilic functional groups each; artisans will immediately appreciate that all the ranges and values within the explicitly stated ranges are contemplated, for instance 2, 3, 4, 5, 6, 7, 8, 9, 10, 11, 12, 13, 14, 15, or 16 groups.

The functional groups may be, e.g., electrophiles reactable with nucleophiles, groups reactable with specific nucleophiles, e.g., primary amines, groups that form amide bonds with materials in the biological fluids, groups that form amide bonds with carboxyls, activated-acid functional groups, or a combination of the same. The functional groups may be, e.g., a strong electrophilic functional group, meaning an electrophilic functional group that effectively forms a covalent bond with a primary amine in aqueous solution at pH 9.0 at room temperature and pressure and/or an electrophilic group that reacts by a of Michael-type reaction. The strong electrophile may be of a type that does not participate in a Michaels-type reaction or of a type that participates in a Michaels-type reaction.

A Michael-type reaction refers to the 1, 4 addition reaction of a nucleophile on a conjugate unsaturated system. The addition mechanism could be purely polar, or proceed through a radical-like intermediate state(s); Lewis acids or appropriately designed hydrogen bonding species can act as catalysts. The term conjugation can refer both to alternation of carbon-carbon, carbon-heteroatom or heteroatom-heteroatom multiple bonds with single bonds, or to the linking of a functional group to a macromolecule, such as a synthetic polymer or a protein. Michael-type reactions are discussed in detail in U.S. Pat. No. 6,958,212, which is hereby incorporated by reference herein in its entirety for all purposes to the extent it does not contradict what is explicitly disclosed herein.

Examples of strong electrophiles that do not participate in a Michaels-type reaction are: succinimides, succinimidyl esters, or NHS-esters. Examples of Michael-type electrophiles are acrylates, methacrylates, methylmethacrylates, and other unsaturated polymerizable groups.

### Initiating Systems

Some precursors react using initiators. An initiator group is a chemical group capable of initiating a free radical polymerization reaction. For instance, it may be present as a separate component, or as a pendent group on a precursor. Initiator groups include thermal initiators, photoactivatable initiators, and oxidation-reduction (redox) systems. Long wave UV and visible light photoactivatable initiators include, for example, ethyl eosin groups, 2,2-dimethoxy-2-phenyl acetophenone groups, other acetophenone derivatives, thioxanthone groups, benzophenone groups, and camphorquinone groups. Examples of thermally reactive initiators include 4,4′ azobis(4-cyanopentanoic acid) groups, and analogs of benzoyl peroxide groups. Several commercially available low temperature free radical initiators, such as V-044, available from Wako Chemicals USA, Inc., Richmond, Va., may be used to initiate free radical crosslinking reactions at body temperatures to form hydrogel coatings with the aforementioned monomers.

Metal ions may be used either as an oxidizer or a reductant in redox initiating systems. For example, ferrous ions may be used in combination with a peroxide or hydroperoxide to initiate polymerization, or as parts of a polymerization system. In this case, the ferrous ions would serve as a reductant. Alternatively, metal ions may serve as an oxidant. For example, the ceric ion (4+ valence state of cerium) interacts with various organic groups, including carboxylic acids and urethanes, to remove an electron to the metal ion, and leave an initiating radical behind on the organic group. In such a system, the metal ion acts as an oxidizer. Potentially suitable metal ions for either role are any of the transition metal ions, lanthanides and actinides, which have at least two readily accessible oxidation states. Particularly useful metal ions have at least two states separated by only one difference in charge. Of these, the most commonly used are ferric/ferrous; cupric/cuprous; ceric/cerous; cobaltic/cobaltous; vanadate V vs. IV; permanganate; and manganic/manganous. Peroxygen containing compounds, such as peroxides and hydroperoxides, including hydrogen peroxide, t-butyl hydroperoxide, t-butyl peroxide, benzoyl peroxide, cumyl peroxide may be used.

An example of an initiating system is the combination of a peroxygen compound in one solution, and a reactive ion, such as a transition metal, in another. In this case, no external initiators of polymerization are needed and polymerization proceeds spontaneously and without application of external energy or use of an external energy source when two complementary reactive functional groups containing moieties interact at the application site.

### Visualization Agents

A visualization agent may be used as a powder in a xerogel/hydrogel; it reflects or emits light at a wavelength detectable to a human eye so that a user applying the hydrogel could observe the object when it contains an effective amount of the agent. Agents that require a machine aid for imaging are referred to as imaging agents herein, and examples include: radioopaque contrast agents and ultrasound contrast agents. Some biocompatible visualization agents are FD&C BLUE #1, FD&C BLUE #2, and methylene blue. These agents are preferably present in the final electrophilic-nucleophilic reactive precursor species mix at a concentration of more than 0.05 mg/ml and preferably in a concentration range of at least 0.1 to about 12 mg/ml, and more preferably in the range of 0.1 to 4.0 mg/ml, although greater concentrations may potentially be used, up to the limit of solubility of the visualization agent. Visualization agents may be covalently linked to the molecular network of the xerogel/hydrogel, thus preserving visualization after application to a patient until the hydrogel hydrolyzes to dissolution. Visualization agents may be selected from among any of the various non-toxic colored substances suitable for use in medical implantable medical devices, such as FD&C BLUE dyes 3 and 6, eosin, methylene blue, indocyanine green, or colored dyes normally found in synthetic surgical sutures. Reactive visualization agents such as NHS-fluorescein can be used to incorporate the visualization agent into the molecular network of the xerogel/hydrogel. The visualization agent may be present with either reactive precursor species, e.g., a crosslinker or functional polymer solution. The preferred colored substance may or may not become chemically bound to the hydrogel.

### Biodegradation

A gel or hydrogel may be formed so that, upon hydration in physiological solution, a gel or hydrogel is formed that is water-degradable, as measurable by the gel or hydrogel losing its mechanical strength and eventually dissipating in vitro in an excess of water by hydrolytic degradation of water-degradable groups. This test is predictive of hydrolytically-driven dissolution in vivo, a process that is in contrast to cell or protease-driven degradation. Significantly, however, polyanhydrides or other conventionally-used degradable materials that degrade to acidic components tend to cause inflammation in tissues. The hydrogels, however, may exclude such materials, and may be free of polyanhydrides, anhydride bonds, or precursors that degrade into acid or diacids. The term degradation by solvation in water, also referred to as dissolving in water, refers to a process of a matrix gradually going into solution in, which is a process that cannot take place for a covalently crosslinked material and materials insoluble in water.

For example, electrophilic groups such as SG (succinimidyl glutarate), SS (succinimidyl succinate), SC (succinimidyl carbonate), SAP (succinimidyl adipate) or SAZ (succinimidyl azelate) may be used and have esteric linkages that are hydrolytically labile. More linear hydrophobic linkages such as pimelate, suberate, azelate or sebacate linkages may also be used, with these linkages being less degradable than succinate, glutarate or adipate linkages. Branched, cyclic or other hydrophobic linkages may also be used. Polyethylene glycols and other precursors may be prepared with these groups. The crosslinked hydrogel degradation may proceed by the water-driven hydrolysis of the biodegradable segment when water-degradable materials are used. Polymers that include ester linkages may also be included to provide a desired degradation rate, with groups being added or subtracted near the esters to increase or decrease the rate of degradation. Thus it is possible to construct a hydrogel with a desired degradation profile, from a few days to many months, using a degradable segment. If polyglycolate is used as the biodegradable segment, for instance, a crosslinked polymer could be made to degrade in about 1 to about 30 days depending on the crosslinking density of the network. Similarly, a polycaprolactone based crosslinked network can be made to degrade in about 1 to about 8 months. The degradation time generally varies according to the type of degradable segment used, in the following order: polyglycolate< polylactate< polytrimethylene carbonate< polycaprolactone. Thus it is possible to construct a hydrogel with a desired degradation profile, from a few days to many months, using a degradable segment. Some embodiments include precursors that are free of adjacent ester groups and/or have no more than one ester group per arm on one or more of the precursors: control of the number and position of the esters can assist in uniform degradation of the hydrogel.

A biodegradable linkage in the organogel and/or xerogel and/or hydrogel and/or gel and/or precursor may be water-degradable or enzymatically degradable. Illustrative water-degradable biodegradable linkages include polymers, copolymers and oligomers of glycolide, dl-lactide, l-lactide, dioxanone, esters, carbonates, and trimethylene carbonate. Illustrative enzymatically biodegradable linkages include peptidic linkages cleavable by metalloproteinases and collagenases. Examples of biodegradable linkages include polymers and copolymers of poly(hydroxy acid)s, poly(orthocarbonate)s, poly(anhydride)s, poly(lactone)s, poly(aminoacid)s, poly(carbonate)s, and poly(phosphonate)s.

If it is desired that a biocompatible crosslinked matrix be biodegradable or absorbable, one or more precursors having biodegradable linkages (or just one biodegradable linkage, for example an ester) present in between the functional groups may be used. The biodegradable linkage optionally also may serve as the water soluble core of one or more of the precursors used to make the matrix. For each approach, biodegradable linkages may be chosen such that the resulting biodegradable biocompatible crosslinked polymer will degrade or be absorbed in a desired period of time.

Hydrogel Loading with Agents; Preparation as Particles

One approach for making a hydrogel or gel or organogel with a therapeutic agent is to form it around the agent. For instance, a first precursor is added to a solvent-protein mixture, followed by a second precursor that is reactive with the first precursor to form crosslinks. After formation of the matrix in the solvent, the solvent may be removed to form a xerogel. Potential processes include, e.g., precipitation with non-solvent, nitrogen sweep drying, vacuum drying, freeze-drying, a combination of heat and vacuum, and lyophilization. If molten precursors are used in the absence of a tertiary solvent, there is no need to employ any solvent removal process. Upon cooling the material forms a rubbery solid (if above Tm), a semirigid semicrystalline material (if below Tm) or a rigid glassy solid (if below Tg). These materials are more dense than xerogels formed from organic solvents. When filled with particles of other materials, e.g., therapeutic agents, buffer salts, visualization agents, they can be highly porous, since the solid particles create and fill the pores.

In some embodiments, the agent or agents are present in a separate phase when precursors are reacted. The separate phase could be oil (oil-in water emulsion), or an immiscible solvent, a liposome, a micelle, a biodegradable vehicle, and the like. Biodegradable vehicles in which the active agent may be present include: encapsulation vehicles, such as microparticles, microspheres, microbeads, micropellets, where the active agent is encapsulated in a bioerodable or biodegradable polymers such as polymers and copolymers of: poly(anhydride), poly(hydroxy acid)s, poly(lactone)s, poly(trimethylene carbonate), poly(glycolic acid), poly(lactic acid), poly(glycolic acid)-co-poly(glycolic acid), poly(orthocarbonate), poly(caprolactone), crosslinked biodegradable hydrogel networks like fibrin glue or fibrin sealant, caging and entrapping molecules, like cyclodextrin, molecular sieves and the like. Microspheres made from polymers and copolymers of poly (lactone) s and poly (hydroxy acid) are particularly preferred as biodegradable encapsulation vehicles. The therapeutic agent or encapsulated therapeutic agent may be present in solution or suspended form. Some agents are highly soluble while others are effectively insoluble in aqueous solution and can form their own phase when exposed to aqueous solvent.

Therapeutic agents can be in solid particulate form in the hydrogel, e.g., as a powder. For instance, water soluble biologics (e.g., proteins) in solid phase can be ground or otherwise formed into a fine powder that is added to the precursors when a matrix is formed. The peptide or other water soluble biologic may be in a solid phase, may be all crystalline, partially crystalline, or essentially free of crystals (meaning more than 90% free of crystals w/w; artisans will immediately appreciate that all the ranges and values within the explicitly stated ranges are contemplated). A powder of a protein refers to a powder made from one or more proteins. Similarly, powders of water soluble biologics are powders having particles made of one or more water soluble biologics. The powders and/or xerogels and/or organogels and/or hydrogels that contain them may be free of encapsulating materials and be free of one or more of a liposome, micelle, or nanocapsule. Further, a protein particle or a water soluble biologic particle may be made that is free of one or more of: binders, non-peptidic polymers, surfactants, oils, fats, waxes, hydrophobic polymers, polymers comprising alkyl chains longer than 4 CH₂ groups, phospholipids, micelle-forming polymers, micelle-forming compositions, amphiphiles, polysaccharides, polysaccharides of three or more sugars, fatty acids, and lipids. Lyophilized, spray dried or otherwise processed proteins are often formulated with sugars such as trehalose to stabilize the protein through the lyophilization or other processes used to prepare the proteins. These sugars may be allowed to persist in the particle throughout the organogel/xerogel process. The particles may be made to comprise between about 20% and about 100% (dry w/w) protein; artisans will immediately appreciate that all the ranges and values within the explicitly stated ranges are contemplated, e.g., about 50% to about 80% or at least 90% or at least about 99%. A number of factors can be controlled that contribute to processing and delivery of a protein without denaturation. The protein may be prepared as a powder, with the powder particle size being chosen in light of the size of the ultimate hydrogel/organogel/xerogel particle. Organic solvents for the proteins may be chosen so that the proteins are not solvated by the organic solvents and are compatible with the protein. Another factor is oxygen, and elimination of oxygen is helpful in processing to avoid denaturation. Another factor is chemical reactions. These may be avoided by keeping the protein in a solid phase and free of solvents that dissolve the protein until such time as the protein is implanted.

A gel or organogel or hydrogel may be formed and then reduced to particles that are subsequently treated to remove the organic or aqueous solvent or solvents to form a xerogel. For an injectable form, the organogel or hydrogel can be macerated, homogenized, extruded, screened, chopped, diced, or otherwise reduced to a particulate form. Alternatively, the organogel or hydrogel can be formed as a droplet or a molded article containing the suspended protein particles. One process for making such particles involves creation of a material that is broken up to make the particles. One technique involves preparing the organogel or hydrogel with protein particles and grinding it, e.g., in a ball mill or with a mortar and pestle. The matrix may be chopped or diced with knives or wires. Or the matrix may be cut-up in a blender or homogenizer. Another process involves forcing the organogel through a mesh, collecting the fragments, and passing them through the same mesh or another mesh until a desired size is reached.

The particles of biologics or the particles of gels or organogels or xerogels may be separated into collections with a desired size range and distribution of sizes by a variety of methods. Very fine control of sizing is available, with sizes ranging from 1 micron to several mm, and with a mean and range of particles sizes being controllable with a narrow distribution. Artisans will immediately appreciate that all the ranges and values within the explicitly stated ranges are contemplated, e.g., from about 1 to about 10 μm or from about 1 to about 30 μm. About 1 to about 500 microns is another such range that is useful, with sizes falling throughout the range and having a mean sizing at one value within the range, and a standard deviation centered around the mean value, e.g., from about 1% to about 100%. A simple method for sizing particles involves using custom-made or standardized sieve mesh sizes. The term particle is broad and includes spheres, discs, and irregularly shaped particles. A spheroidal particle refers to a particle wherein the longest central axis (a straight line passing through the particle's geometric center) is no more than about twice the length of other central axes, with the particle being a literally spherical or having an irregular shape. A rod-shaped particle refers to a particle with a longitudinal central axis more than about twice the length of the shortest central axis. Embodiments include making a plurality of collections of particles, with the collections having different rates of degradation in vivo, and mixing collections to make a biomaterial having a degradation performance as desired.

### The Eye

Administration of a fluent material, e.g., a precursor to form a hydrogel may be performed directly into the site of interest. Embodiments of the invention include administration at or near an eye 100, at FIGS. 1-2. The structure of the mammalian eye can be divided into three main layers or tunics: the fibrous tunic, the vascular tunic, and the nervous tunic. The fibrous tunic, also known as the tunica fibrosa oculi, is the outer layer of the eyeball consisting of the cornea 102 and sclera 104. Sclera 104 extends from cornea 102 (the clear front section of the eye) to optic nerve 106 at the back of the eye. The sclera is a fibrous, elastic and protective tissue, composed of tightly packed collagen fibrils, containing about 70% water. Overlaying the fibrous tunic is conjunctiva 108. The conjunctiva is a membrane that covers the sclera (white part of the eye) and lines the inside of the eyelids. The conjunctiva is typically divided into three parts: (a) Palpebral or tarsal conjunctiva which is the conjunctiva lining the eyelids; the palpebral conjunctiva is reflected at the superior fornix and the inferior fornix to become the bulbar conjunctiva; (b) Fornix conjunctiva: the conjunctiva where the inner part of the eyelids and the eyeball meet; and (c) Bulbar or ocular conjunctiva: The conjunctiva covering the eyeball, over the sclera. This region of the conjunctiva is bound tightly and moves with the eyeball movements. The conjunctiva effectively surrounds, covers, and adheres to the sclera. It is has cellular and connective tissue, is somewhat elastic, and can be removed, teased away, or otherwise taken down to expose a surface area of the sclera.

The vascular tunic, also known as the tunica vasculosa oculi, is the middle vascularized layer which includes the iris, ciliary body, and choroid 110. Choroid 110 lies between retina 112 and sclera 104. The choroid contains blood vessels that supply the retinal cells with oxygen and remove the waste products of respiration. The choroid connects with the ciliary body toward the front of the eye and is attached to edges of optic nerve 106 at the back of the eye. The nervous tunic, also known as the tunica nervosa oculi, is the inner sensory which includes the retina. The retina contains the photosensitive rod and cone cells and associated neurons. The retina is a relatively smooth (but curved) layer. It does have two points at which it is different; fovea 114 and optic disc. The fovea is a dip in the retina directly opposite the lens, which is densely packed with cone cells. The fovea is part of the macula. The optic disc is a point on the retina where the optic nerve pierces the retina to connect to the nerve cells on its inside. The mammalian eye can also be divided into two main segments: the anterior segment and the posterior segment. The anterior segment consists of an anterior and posterior chamber.

The cornea and lens help to converge light rays to focus onto the retina. The lens, behind the iris, is a convex, springy disk which focuses light, through the second humour, onto the retina. It is attached to the ciliary body via a ring of suspensory ligaments known as the Zonule of Zinn. The iris, between the lens and the first humour, is a pigmented ring of fibrovascular tissue and muscle fibers. Light must first pass though the center of the iris, the pupil. Light enters the eye, passes through the cornea, and into the first of two humors, the aqueous humour. Approximately two-thirds of the total eyes refractive power comes from the cornea which has a fixed curvature. The aqueous humor is a clear mass which connects the cornea with the lens of the eye, helps maintain the convex shape of the cornea (necessary to the convergence of light at the lens) and provides the corneal endothelium with nutrients. The posterior segment is posterior to the crystalline lens and in front of the retina. It includes the anterior hyaloid membrane and the structures behind it, including the vitreous humor, retina, and optic nerve.

In use, for example a syringe, catheter or other device is used to deliver precursors. When precursors are delivered, they are chosen so they form hydrogel in situ at the site of intended use. The therapeutic agents are released from the hydrogels. Various sites may be chosen. Sites where drug delivery depots may be formed include the anterior chamber, posterior chamber, the vitreous, episcleral, subconjunctival, on a surface of a cornea or a conjunctiva, on a sclera, in a sclera, beneath a sclera, or between a sclera and subconjunctiva in a site under and contacting the conjunctiva, on or under the palpebral or tarsal conjunctiva, in an eyelid, superior fornix, inferior fornix, bulbar conjunctiva, and fornix conjunctiva. Further sites are in the choroid, between the choroid and sclera, between the retina and choroid, or a combination of the same.

The hydrogel may be placed at a site that is suited to deliver the agent for the pathology that is being treated. The choice of dose, size of implant, and position is affected by factors such as a time between repeat administrations, patient comfort or compliance, and dosage received at a target tissue. In general, back of the eye diseases can be treated with drugs utilizing, e.g., topical, systemic, intraocular and subconjunctival delivery routes. Systemic and topical (referring to eye drops and non-adherent materials) delivery modalities fall short in delivering therapeutic drug levels to treat posterior segment diseases: these methods of drug delivery encounter diffusion and drug dilution issues due to the inherent anatomical barriers of the intraocular and systemic systems, causing significant patient side effects (due to multiple daily dosing), poor bioavailability and compliance issues. Pericular drug delivery of an ophthalmic hydrogel implant using subconjunctival, scleral, retrobulbar or sub-Tenon's placement has the potential to offer a safer and enhanced drug delivery system to the retina compared to topical and systemic routes. For example; steroids like dexamethasone and triamicinolone acetonide may be mixed with the hydrogel precursor to form a sustained-release drug implant. The liquid hydrogel could then be injected in situ into the sub-Tenon's capsule where it could deliver a constant or tunable release profile of the drug over a three to four month time period. The minimally invasive procedure could be performed in a doctor's office, or after a cataract operation under topical anesthesia, to treat chronic back of the eye diseases.

In some embodiments, a retractor is used to hold back eyelids, the user create a small buttonhole in the conjunctiva about 5-6 mm from the inferior/nasal limbus and dissects the conjunctiva down through Tenon's capsule, to the bare sclera. Next, a 23-gauge blunt cannula 86 (e.g., 15 mm in length) is inserted through the opening and the liquid drug implant is injected at the intended site of use. The cannula is then removed and the conjunctive is closed with a cauterization device. One advantage of a hydrogel implant having three dimensional integrity is that it will tend to resist cellular infiltration and be able to prevent the locally administered drug from being phagocytosed and cleared prematurely from the site. Instead, it stays in place until delivered. By way of contrast a microparticle, liposome, or pegylated protein tends to be rapidly cleared from the body by the reticuloendothelial system before being bioeffective.

Delivery of therapeutic amounts of a drug to the retina in posterior segment eye diseases remains a challenge. Although intravitreal injections into the vitreous cavity of antiangiogenesis agents, such as anti-VEGF, have shown promise to arrest and in some cases reverse chronic age-related diseases like macular degeneration, these techniques and procedures are not without risks and side effects. Intravitreal administration of therapeutic agents into the vitreous cavity can cause cataracts, endophthalmitis and retinal detachments. This form of therapy requires many patients to receive monthly intraocular injections of an anti-VEGF drug thus increasing the risk of infection, vitreous wicks and retinal detachments. Embodiments directed to an in situ hydrogel biodegradable drug implant that contains hydrogel particles will provide an effective alternative treatment for back of the eye diseases, and are expected to reduce the common side-effects associated with repeated intravitreal injections. For intravitreal implacement, for example, a hydrogel precursors and hydrogel particles are injected intravitrealy about 2.5 mm posterior to the limbus through a pars plana incision using a sub-retinal cannula, which may be made following dissecting-away or otherwise clearing the conjunctiva, as needed. A 25, 27 or 30 gauge sub-retinal cannula 94 (or other appropriate cannula) is then inserted and positioned intraocularly to the desired target site where the flowable precursors are introduced to form a hydrogel in situ. The precursors then forms into an absorbable gel, adhering to the desired target site.

A drug depot of the in situ hydrogel drug delivery implant may be designed for controlled, long term drug release ranging from, e.g., about one to about 12 months; and may optionally be directed to treatment of diseases of the posterior segment including, for example, age-related macular degeneration, diabetic retinopathy, diabetic macular edema, and the cystoid macular. The device can carry a drug payload of various types of therapeutic agents for various conditions, of which some include, for example, steroids, antibiotics, NSAIDS and/or antiangiogenic agents, or combinations thereof. The in situ implant embodiments can improve the efficacy and pharmacokinetics of potent therapeutic agents in the treatment of chronic back of the eye diseases and minimize patient side effects in several ways. First, the implant can be placed in the vitreous cavity at a specific disease site, bypassing the topical or systemic routes and thereby increasing drug bioavailability. Secondly, the implant maintains local therapeutic concentrations at the specific target tissue site over an extended period of time. Thirdly, as compared to various conventional systems, the number of intravitreal injections would be substantially reduced, thereby reducing patient risk of infection, retinal detachment and transient visual acuity disturbances (white specks floating in the vitreous) that can occur until the drug in the vitreous migrates down toward the inferior wall of the eye and away from the portion of the central vitreous or macula. A bolus of conventionally-injected drugs forms in the vitreous body and displaces the vitreous humor until dispersed. Dispersion typically takes a significant amount of time since the vitreous humor is quite viscous. The bolus thus interferes with vision, particularly when it is moved around the eye in response to sudden accelerations, e.g., as the patient stands up or quickly turns the head.

The hydrogels may be formed in, on, or under scleral tissue either with or without the presence of the conjunctiva. The hydrogel may be adhesive to the sclera or other tissue where it is placed to promote drug diffusion through the intended tissue or to provide a stable depot to direct the therapeutic agents as required. In some embodiments, the conjunctiva of the eye may be removed, macerated, dissected away, or teased-free so that the tissue can be lifted away from the sclera to access a specific region of the sclera for implantation or injection of the hydrogel. In other embodiments, the hydrogel is injected in or on the choroid. A hydrogel is formed in situ that makes a layer on, and adheres, to the target site. In some embodiments the hydrogel is comprised of at least 50%, 75%, 80%, 90%, or 99% w/w water-soluble precursors (calculated by measuring the weight of the hydrophilic precursors and dividing by the weight of all precursors, so that the weight of water or solvents or non-hydrogel components is ignored) to enhance the non-adhesive properties of the hydrogel. In some embodiments, such hydrophilic precursors substantially comprise polyethylene oxides. In some embodiments, drugs to reduce tissue adherence mediated by biological mechanisms including cell mitosis, cell migration, or macrophage migration or activation, are included, e.g., anti-inflammatories, anti-mitotics, antibiotics, PACLITAXEL, MITOMYCIN, or taxols.

In some embodiments, the conjunctiva may be punctured or penetrated with a needle or catheter or trocar and precursors introduced into a space between the sclera and conjunctiva or other spaces in the eye. In some cases the conjunctiva may be punctured to access a natural potential space between the tissues that is filled by the precursors, for instance a choroid. In other cases, a potential or actual space is created mechanically with a trocar, spreader, or the like, that breaks the adherence between the sclera and conjunctiva so that precursors may be introduced. The conjunctiva has enough elasticity to allow useful amounts of precursors to be introduced or forced into such natural or created spaces. Similarly, in the case of intravitreal hydrogel formation, relatively large columns may also be used. Accordingly, in some cases, referring generally to sites in or around an eye, the amount may be quite small to relatively large, e.g., an amount from about 0.001 to about 10 ml; artisans will immediately appreciate that all the ranges and values within the explicitly stated ranges are contemplated, e.g., about 1, 2, 3, 4, 5 6, 7, 8, 9, 10, 20, 30, 40, 50, 100 microliters or 0.2, 0.3, 0.4, 0.5, 1, 2, 3, 4, 5, 6, 7, 8, 9, or 10 ml.

In some aspects, in situ formation of the hydrogel lets the hydrogel gel or crosslink in place, so that it does not flow back out through the tract of the needle and diffuse back out through the incision site upon the removal of the needle or cannula. A shape-stable hydrogel thus formed can effectively deliver the drug and advantageously can have well-controlled size, shape, and surface area. A small needle may be used to inject the materials since soluble or flowable precursors may be used instead of an already-formed material. By way of contrast, alternative materials that do not cross-link quickly and firmly upon introduction tend to flow back out of the incision. And materials that do not covalently cross-link are subject to creep or weeping as the material continually reorganizes and some or all of the material flows out.

### Delivery to a Tissue

The delivery devices may be used to deliver their contents to any site, including any soft or hard tissue, and placement, e.g., intramuscular, intraperitoneal, subcutaneous. Their contents may be a precursor or agent as set forth herein, or other materials.

### Kits

Kits or systems for making gels or hydrogels may be prepared so that the precursor(s) and therapeutic agent(s) are stored in the kit with diluents as may be needed. Applicators may be used in combination with the same. The kits are manufactured using medically acceptable conditions and contain components that have sterility, purity and preparation that is pharmaceutically acceptable. Solvents/solutions, diluents may be provided in the kit or separately, or the components may be pre-mixed with the solvent. The kit may include syringes and/or needles for mixing and/or delivery. The kit or system may comprise components set forth herein. Instructions for carrying out one or methods set forth herein may be provided.

### Administration

An embodiment is a hydrogel formed by in situ polymerization containing a therapeutic agent. In use, precursors and the agent(s) and injected into the site of intended use in the patient. The precursors react with each other to form the hydrogel. A needle, cannula, trocar, sprayer, or other applicator may be used. Administration of the hydrogels and/or xerogels may also involve hydration in advance, at about the time of use, or at the point of use.

The materials described herein may be used to deliver drugs or other therapeutic agents (e.g., imaging agents or markers). One mode of application is to apply a mixture of precursors and other materials (e.g., therapeutic agent, buffer, accelerator, initiator) through a needle, microneedle, cannula, catheter, or hollow wire to a site. The mixture may be delivered, for instance, with a device as set forth herein, with a manually controlled syringe, or with mechanically controlled syringe, e.g., a syringe pump. Alternatively, a dual syringe or multiple-barreled syringe or multi-lumen system may be used to mix the materials at or near the site with a hydrating fluid and/or other agents. Fine needles may be used and/or needles with a limited length. The work may be performed, if helpful, under magnification, with a stereoscope, with guided imaging, or with robots (for instance as described by Eindhoven University of Technology).

### EXAMPLES

### Example 1

### Preparation and Injection of Hydrogels into the Suprachoroidal Space

In a 20 mL serum vial, dissolved 171.97 mg Trilysine Acetate in 15 mL of 0.175M Sodium Phosphate Dibasic solution. Transferred solution into a 25 mL graduated cylinder and added 0.175M Sodium Phosphate Dibasic solution using a transfer pipette to bring final volume to 18 mL. The resulting solution was then transferred back into the original 20 mL serum vial.

Weighed 57.60±0.5 mg NHS-Fluorescein (5(6)-Carboxyfluorescein, Succinimidyl Ester) (NHS-Fluorescein) and transferred to the vial containing the Trilysine Acetate/Sodium Phosphate Dibasic solution and vortexed until completely dissolved. The vial was immediately wrapped in foil to prevent light exposure and allowed to react for 1 hour.

After 1 hour, 0.25 mL of the Trilysine Acetate/NHS-Fluorescein/Sodium Phosphate Dibasic solution was aliquotted into a 5 mL syringe.

In a second 5 mL syringe, 125 mg of Dexamethasone, Micronized, USP was weighed. Using a volumetric pipette, 0.5 mL of DI water was added to the syringe. The syringe plunger was then replaced and the syringe was vortexed to suspend the Dexamethasone.

Weighed 90 mg of 4-armed 20 KDa polyethylene glycol (PEG) end capped with succinimidylsuccinate groups (4a20kSG PEG) powder and transferred into a third 5 mL syringe, then added 0.25 mL of 0.075M Sodium Phosphate Monobasic solution to dissolve the 4a20kSG PEG.

The contents of the 4a20kSG PEG/Sodium Phosphate Monobasic solution syringe and the Dexamethasone suspension syringe were combined by mating the two syringes with a luer-luer connector and passing the contents back and forth between the two syringes for approximately 10 seconds. The resulting mixture was then drawn into one syringe. The combined mixture created in the preceding step was then mixed with the Trilysine/NHS-Fluorescein/Sodium Phosphate Dibasic solution syringe using the same mixing method. This final mixing step initiated the crosslinking reaction, and a stopwatch was started at the onset of this final mixing step.

Approximately 200 μL of the hydrogel precursor solution was introduced into each of three 250 μL Hamilton Glass Syringe. Each was then expelled through a microneedle of approximately 600 μm length and varying in inner diameter from 120 μm to 160 μm. The contents of each syringe were expelled freely and completely without visible signs of the micronized suspension remaining in the syringe or otherwise being obstructed by the small diameter of the needle.

### Repeat Same Preparation, Larger Volume, Injected Suprachoroidally:

0.5 mL of the previously prepared Trilysine Acetate/NHS-Fluorescein/Sodium Phosphate Dibasic solution was aliquotted into a 5 mL syringe.

In a second 5 mL syringe, 250 mg of Dexamethasone, Micronized, USP was weighed. Using a volumetric pipette, 1.0 mL of DI water was added to the syringe. The syringe plunger was then replaced and the syringe was vortexed to suspend the Dexamethasone.

Weighed 180 mg of 4a20kSG PEG powder and transferred into a third 5 mL syringe, then added 0.5 mL of 0.075M Sodium Phosphate Monobasic solution to dissolve the PEG.

The contents of the 4a20kSG PEG/Sodium Phosphate Monobasic solution syringe and the Dexamethasone suspension syringe were combined by mating the two syringes with a luer-luer connector and passing the contents back and forth between the two syringes for approximately 10 seconds. The resulting mixture was then drawn into one syringe. The combined mixture created in the preceding step was then mixed with the Trilysine/NHS-Fluorescein/Sodium Phosphate Dibasic solution syringe using the same mixing method. This final mixing step initiated the crosslinking reaction, and a stopwatch was started at the onset of this final mixing step.

Approximately 200 μL of the hydrogel precursor solution was introduced into a 250 μL Hamilton Glass Syringe. The mixture was then injected into the suprachoroidal space of an ex-vivo porcine eye through a microneedle of approximately 600 μm length and varying in inner diameter from 120 μm to 160 μm. The remainder of the solution was then expelled onto a glass slide and the time until the material gelled and became a solid material was observed on the stopwatch. The hydrogel was allowed to continue crosslinking in the ex-vivo eye for an additional 15 minutes.

Following the 15 minute hold to allow additional crosslinking, UBM imaging of the injection location was performed and indicated that the hydrogel was delivered to the suprachoroidal space. The eye was then dissected and a cross-section of the injection location was isolated. The hydrogel was observed in the cross-section to be a layer, yellow in color, <1 mm in thickness between the choroid and the sclera. The hydrogel was observed to be distributed in approximately a 2.5 mm radius around the injection site and in a relatively uniform pattern. A cobalt blue light was used to excite the NHS-Fluorescein and was observed through yellow glasses to facilitate visualization of the fluoresceinated hydrogel in the tissue sample.

Additional injections into the suprachoroidal space of ex-vivo porcine eyes were performed using the same formulation and the same size needle. A total of four injections were performed, varying in volume from 50 μL to 150 μL. Each was confirmed to be a uniformly distributed hydrogel in the suprachoroidal space following injection.

Applications include ocular implants for the treatment of conditions such as idiopathic uveitis, corneal transplantation, dry eye syndrome, age-related macular degeneration (AMD, wet and dry), diabetic eye conditions, blepharitis, glaucoma, ocular hypertension, post-operative eye pain and inflammation, posterior segment neovascularization (PSNV), proliferative vitreoretinopathy (PVR), cytomegalovirus retinitis (CMV), endophthalmitis, choroidal neovascular membranes (CNVM), vascular occlusive diseases, allergic eye disease, tumors, retinitis pigmentosa, eye infections, scleritis, ptosis, miosis, eye pain, mydriasis, neuralgia, aging (e.g. muscle relaxants and other aesthetic products), cicatrizing ocular surface diseases, ocular infections, inflammatory ocular diseases, ocular surface diseases, corneal diseases, retinal diseases, ocular manifestations of systemic diseases, hereditary eye conditions, ocular tumors, and increased intraocular pressure. Applications include diabetic retinopathy, chronic glaucoma, retinal detachment, sickle cell retinopathy, retinal neovascularization, subretinal neovascularization; rubeosis irides, retinitis, choroiditis, posterior uveitis, neoplasms, retinoblastoma, pseudoglioma, neovascular glaucoma; neovascularization (for instance, resulting from vitrectomy and/or lensectomy), vascular diseases, retinal ischemia, choroidal vascular insufficiency, choroidal thrombosis, neovascularization of the optic nerve, diabetic macular edema, cystoid macular edema, macular edema, retinal vein occlusion, angioid streaks, retinal artery occlusion, and neovascularization due to ocular injury.

Therapeutic agents for use may include, for instance, steroids, non-steroidal anti-inflammatory drugs (NSAIDS), anti-cancer drugs, antibiotics, an anti-inflammatory (e.g., Diclofenac), a pain reliever (e.g., Bupivacaine), a Calcium channel blocker (e.g., Nifedipine), an Antibiotic (e.g., Ciprofloxacin), a Cell cycle inhibitor (e.g., Simvastatin), a protein (e.g., Insulin). Therapeutic agents include classes of drugs including steroids, NSAIDS, antibiotics, pain relievers, inhibitors of vascular endothelial growth factor (VEGF), chemotherapeutics, anti-viral drugs, for instance. Examples of NSAIDS are Ibuprofen, Meclofenamate sodium, mefanamic acid, salsalate, sulindac, tolmetin sodium, ketoprofen, diflunisal, piroxicam, naproxen, etodolac, flurbiprofen, fenoprofen calcium, Indomethacin, celoxib, ketrolac, and nepafenac. The drugs themselves may be small molecules, peptides, proteins, RNA fragments, glycosaminoglycans, carbohydrates, nucleic acid, inorganic and organic biologically active compounds where specific biologically active agents include but are not limited to: enzymes, antibiotics, antineoplastic agents, local anesthetics, hormones, angiogenic agents, anti-angiogenic agents, growth factors, antibodies, neurotransmitters, psychoactive drugs, anticancer drugs, chemotherapeutic drugs, drugs affecting reproductive organs, genes, and oligonucleotides, or other configurations.

Therapeutic agents may include a protein or other water soluble biologics. These include peptides and proteins. The term protein, as used herein, refers to peptides of at least about 5000 Daltons. The term peptide, as used herein, refers to peptides of any size. The term oligopeptide refers to peptides having a mass of up to about 5000 Daltons. Peptides include therapeutic proteins and peptides, antibodies, antibody fragments, short chain variable fragments (scFv), growth factors, angiogenic factors, and insulin. Other water soluble biologics are carbohydrates, polysaccharides, nucleic acids, antisense nucleic acids, RNA, DNA, small interfering RNA (siRNA), and aptamers.

The therapeutic agents may be used as part of a method of treating the indicated condition or making a composition for treating the indicated condition. For example, AZOPT (a brinzolamide opthalmic suspension) may be used for treatment of elevated intraocular pressure in patients with ocular hypertension or open-angle glaucoma. BETADINE in a Povidone-iodine ophthalmic solution may be used for prepping of the periocular region and irrigation of the ocular surface. BETOPTIC (betaxolol HCl) may be used to lower intraocular pressure, or for chronic open-angle glaucoma and/or ocular hypertension. CILOXAN (Ciprofloxacin HCl opthalmic solution) may be used to treat infections caused by susceptible strains of microorganisms. NATACYN (Natamycin opthalmic suspension) may be used for treatment of fungal blepharitis, conjunctivitis, and keratitis. NEVANAC (Nepanfenac opthalmic suspension) may be used for treatment of pain and inflammation associated with cataract surgery. TRAVATAN (Travoprost ophthalmic solution) may be used for reduction of elevated intraocular pressure-open-angle glaucoma or ocular hypertension. FML FORTE (Fluorometholone ophthalmic suspension) may be used for treatment of corticosteroid-responsive inflammation of the palperbral and bulbar conjunctiva, cornea and anterior segment of the globe. LUMIGAN (Bimatoprost ophthalmic solution) may be used for reduction of elevated intraocular pressure-open-angle glaucoma or ocular hypertension. PRED FORTE (Prednisolone acetate) may be used for treatment of steroid-responsive inflammation of the palpebral and bulbar conjunctiva, cornea and anterior segment of the globe. PROPINE (Dipivefrin hydrochloride) may be used for control of intraocular pressure in chronic open-angle glaucoma. RESTASIS (Cyclosporine ophthalmic emulsion) may be used to increases tear production in patients, e.g., those with ocular inflammation associated with keratoconjunctivitis sicca. ALREX (Loteprednol etabonate ophthalmic suspension) may be used for temporary relief of seasonal allergic conjunctivitis. LOTEMAX (Loteprednol etabonate ophthalmic suspension) may be used for treatment of steroid-responsive inflammation of the palpebral and bulbar conjunctiva, cornea and anterior segment of the globe. MACUGEN (Pegaptanib sodium injection) may be used for Treatment of neovascular (wet) age-related macular degeneration. OPTIVAR (Azelastine hydrochloride) may be used for treatment of itching of the eye associated with allergic conjunctivitis. XALATAN (Latanoprost ophthalmic solution) may be used to reduce elevated intraocular pressure in patients, e.g., with open-angle glaucoma or ocular hypertension. BETIMOL (Timolol opthalmic solution) may be used for treatment of elevated intraocular pressure in patients with ocular hypertension or open-angle glaucoma. Latanoprost is the pro-drug of the free acid form, which is a prostanoid selective FP receptor agonist. Latanoprost reduces intraocular pressure in glaucoma patients with few side effects. Latanoprost has a relatively low solubility in aqueous solutions, but is readily soluble in organic solvents typically employed for fabrication of microspheres using solvent evaporation.

Further embodiments of therapeutic agents for delivery include those that specifically bind a target peptide in vivo to prevent the interaction of the target peptide with its natural receptor or other ligands. AVASTIN, for instance, contains bevacizumab, an antibody that binds VEGF. And AFLIBERCEPT is a fusion protein that includes portions of a VEGF receptor to trap VEGF. An IL-1 trap that makes use of the extracellular domains of IL-1 receptors is also known; the trap blocks IL-1 from binding and activating receptors on the surface of cells. Embodiments of agents for delivery include nucleic acids, e.g., aptamers. Pegaptanib (MACUGEN), for example, is a pegylated anti-VEGF aptamer. An advantage of the particle-and-hydrogel delivery process is that the aptamers are protected from the in vivo environment until they are released. Further embodiments of agents for delivery include macromolecular drugs, a term that refers to drugs that are significantly larger than classical small molecule drugs, i.e., drugs such as oligonucleotides (aptamers, antisense, RNAi), ribozymes, gene therapy nucleic acids, recombinant peptides, and antibodies.

One embodiment comprises extended release of a medication for allergic conjunctivitis. For instance, ketotifen, an antihistamine and mast cell stabilizer, may be provided in particles and released to the eye as described herein in effective amounts to treat allergic conjunctivitis. Seasonal Allergic Conjunctivitis (SAC) and Perennial Allergic Conjunctivitis (PAC) are allergic conjunctival disorders. Symptoms include itching and pink to reddish eyes. These two eye conditions are mediated by mast cells. Non-specific measures to ameliorate symptoms conventionally include: cold compresses, eyewashes with tear substitutes, and avoidance of allergens. Treatment conventionally consists of antihistamine mast cell stabilizers, dual mechanism anti-allergen agents, or topical antihistamines. Corticosteroids might be effective but, because of side effects, are reserved for more severe forms of allergic conjunctivitis such as vernal keratoconjunctivitis (VKC) and atopic keratoconjunctivitis (AKC). Oxifloxacin is the active ingredient in VIGAMOX, which is a fluoroquinolone approved for use to treat or prevent ophthalmic bacterial infections. Dosage is typically one-drop of a 0.5% solution that is administered 3 times a day for a period of one-week or more. VKC and AKC are chronic allergic diseases where eosinophils, conjunctival fibroblasts, epithelial cells, mast cells, and/or TH2 lymphocytes aggravate the biochemistry and histology of the conjunctiva. VKC and AKC can be treated by medications used to combat allergic conjunctivitis. Permeation agents are agents and may also be included in a gel, hydrogel, organogel, xerogel, and biomaterials as described herein. These are agents that assist in permeation of a drug into an intended tissue. Permeation agents may be chosen as needed for the tissue, e.g., permeation agents for skin, permeation agents for an eardrum, and permeation agents for an eye.

### Drugs and Conditions: FDA Approved Drugs for Ophthalmology

### Drugs Approved in 2014

Hetlioz (tasimelteon); Vanda Pharmaceuticals; For the treatment of non-24-hour sleep-wake disorder in the totally blind, January 2014.

Oralair (Sweet Vernal, Orchard, Perennial Rye, Timothy and Kentucky Blue Grass Mixed Pollens Allergen Extract); Greer Labs; For the treatment of grass pollen-induced allergic rhinitis with or without conjunctivitis, Approved April 2014.

### Drugs Approved in 2012

Cystaran (cysteamine hydrochloride); Sigma Tau Pharmaceuticals; For the treatment of corneal cystine crystal accumulation due to cystinosis, Approved October 2012.

Jetrea (ocriplasmin); Thrombogenics; For the treatment of symptomatic vitreomacular adhesion, Approved October 2012.

Lucentis (ranibizumab injection); Genentech; For the treatment of diabetic macular edema, Approved August 2012.

Zioptan (tafluprost ophthalmic solution); Merck; For the treatment of elevated intraocular pressure, Approved February 2012.

### Drugs Approved in 2011

Eylea (aflibercept); Regeneron Pharmaceuticals; For the treatment of neovascular (wet) age-related macular degeneration, Approved November 2011.

### Drugs Approved in 2010

Zymaxid (gatifloxacin ophthalmic solution); Allergan; For the treatment of bacterial conjunctivitis, Approved May 2010.

### Drugs Approved in 2009

Acuvail (ketorolac tromethamine); Allergan; For the treatment of pain and inflammation following cataract surgery, Approved July 2009.

Bepreve (bepotastine besilate ophthalmic solution); Ista Pharmaceuticals; For the treatment of itching associated with allergic conjunctivitis, Approved September 2009.

Besivance (besifloxacin ophthalmic suspension); Bausch & Lomb; For the treatment of bacterial conjunctivitis, Approved June 2009.

Ozurdex (dexamethasone); Allergan; For the treatment of macular edema following branch retinal vein occlusion or central retinal vein occlusion, Approved June 2009.

Zirgan (ganciclovir ophthalmic gel); Sirion Therapeutics; For the treatment of acute herpetic keratitis, Approved September 2009.

Durezol (difluprednate); Sirion Therapeutics; For the treatment of inflammation and pain associated with ocular surgery, Approved June 2008.

### Drugs Approved in 2007

AzaSite (azithromycin); InSite Vision; For the treatment of bacterial conjunctivitis, Approved April 2007.

### Drugs Approved in 2006

Lucentis (ranibizumab); Genentech; For the treatment of neovascular (wet) age related macular degeneration, Approved June 2006.

### Drugs Approved in 2004

Macugen (pegaptanib); Pfizer/Eyetech Pharmaceuticals; For the treatment of wet age-related macular degeneration, Approved December 2004.

### Drugs Approved in 2001

Lumigan (bimatoprost ophthalmic solution); Allergan; For the reduction of intraocular pressure in patients with open-angle glaucoma or ocular hypertension, Approved March 2001.

Travatan (travoprost ophthalmic solution); Alcon; For the reduction of elevated intraocular pressure in patients with open-angle glaucoma or ocular hypertension, Approved March 2001.

Valcyte (valganciclovir HCl); Roche; For the treatment of cytomegalovirus retinitis in patients with AIDS, Approved March 2001.

### Drugs Approved in 2000

Betaxon; Alcon; For lowering IOP in patients with chronic open-angle glaucoma or ocular hypertension, Approved February 2000.

Quixin (levofloxacin); Santen; For treatment of bacterial conjunctivitis, Approved August 2000.

Rescula (unoprostone isopropyl ophthalmic solution) 0.15%; Ciba Vision; For the treatment of open-angle glaucoma or ocular hypertension, Approved August 2000.

Visudyne (verteporfin for injection); QLT; For the treatment of wet age-related macular degeneration (wet AMD), Approved April 2000.

### Drugs Approved in 1999

Alamast; Santen; pemirolast potassium ophthalmic solution, Approved September 1999.

ZADITOR; Ciba Vision; Treatment for the prevention of itching of the eye, Approved July 1999.

### Drugs Approved in 1998

Alrex; Bausch & Lomb, Pharmos; Treatment for seasonal allergic conjunctivitis, Approved March 1998.

Cosopt; Merck; Treatment for glaucoma or ocular hypertension, Approved April 1998.

Lotemax; Bausch & Lomb, Pharmos; Treatment for post-operative eye inflammation, Approved March 1998.

Salagen Tablets; MGI Pharma; Treatment for Sjogren's Syndrome, Approved February 1998.

Viroptic; King Pharmaceuticals; Treatment for inflammation of the cornea in children due to herpes simplex virus, Approved February 1998.

Vitravene Injection; Isis Pharmaceuticals; Treatment for CMV in AIDS patients, Approved August 1998.

### Drugs Approved in 1997

Acular (ketorolac tromethamine ophthalmic solution) 0.5%; Allergan; Treatment for postoperative inflammation in patients who have undergone cataract extraction, Approved January 1997.

Acular (ketorolac tromethamine ophthalmic solution) 0.5%; Allergan; Treatment for post-surgical inflammation following cataract extraction, Approved November 1997.

BSS Sterile Irrigating Solution; Alcon; Treatment during ocular surgical procedures, Approved December 1997.

### Drugs Approved in 1996

AK-Con-A (naphazoline ophthalmic); Akorn; Over-the-counter combination vasoconstrictor/antihistamine product for opthalmic use, Approved January 1996.

Alphagan (brimonidine); Allergan; Treatment for open-angle glaucoma and ocular hypertension, Approved September 1996.

Ocuflox (ofloxacin opthalmic solution) 0.3%; Allergan; Treatment for corneal ulcers, Approved May 1996.

OcuHist; Pfizer; Over-the-counter antihistamine eye drop, Approved January 1996.

Vistide (cidofovir); Gilead; Treatment for cytomegalovirus (CMV) retinitis, Approved June 1996.

### Further Disclosure

1. A method of treating an ophthalmic pathology affecting an eye of a patient comprising forming, in situ, a continuous cohesive layer of covalently-crosslinked hydrogel at a choroid in the eye, wherein the hydrogel comprises a therapeutic agent that is released into the eye to treat the ophthalmic pathology.

2. The method of 1 comprising introducing a hydrogel precursor in aqueous solution at a(n injection) site at the choroid that flows from the site and reacts to form the hydrogel.

3. The method of 2 wherein the precursor is a first precursor, further comprising a second precursor in the aqueous solution, with the first precursor and second precursor crosslinking with each other to form the hydrogel.

4. The method of any of 1-3 wherein the solution has a first pH when introduced at the choroid and a second pH after the introduction, with the precursor being reactive at the second pH.

5. The method of any of 1-4 further comprising an initiator chemical in the aqueous solution, with the initiator initiating reaction of the precursor to form the hydrogel.

6. The method of any of 1-5 wherein the layer has a height from about 0.1 mm to about 2 mm.

7. The method any of 1-6 wherein the layer contacts the choroid in an area sized from about 2 to about 60 mm².

8. The method any of 1-7 wherein the layer has a height from about 0.1 mm to about 2 mm and an area of contact with the choroid from about 12 to about 60 mm².

9. The method any of 1-8 wherein the layer has a discoidal shape with a height from about 0.1 to about 2 mm and an area in contact with the choroid having a radius at least twice the height.

10. The method any of 1-9 wherein the hydrogel, when formed, has a volume of between about 1 μl and about 1 ml.

11. The method any of 1-10 wherein the layer further comprises a plurality of particles that comprise the therapeutic agent.

12. The method of 11 further comprising an additional amount of the same or a different therapeutic agent that is not in the particles and is in the hydrogel.

13. The method of 11 wherein the particles are hydrogels that comprise the agent, which is in a solution, is a solid, or is in suspension.

14. The method of 11 wherein the particles are solid and comprise the agent.

15. The method of 11 wherein the particle is selected from the group consisting of a liposome, a micelle, and a hydrophobic drop.

16. The method any of 1-10 comprising the therapeutic agent as a suspension in an aqueous phase of the hydrogel.

17. The method of any of 1-16 wherein the hydrogel is low-swelling, as measurable by the hydrogel having a weight increasing no more than about 50% upon exposure to a physiological solution for twenty-four hours relative to a weight of the hydrogel at the time of formation.

18. The method any of 1-17 wherein the hydrogel is water-degradable, as measurable by the hydrogel being dissolvable in vitro in an excess of water by degradation of water-degradable groups.

19. The method of 17 wherein the water-degradable groups are esters.

20. The method any of 1-19 wherein the hydrogel is formed by combining a first synthetic precursor comprising nucleophilic groups with a second synthetic precursor comprising electrophilic groups to form covalent crosslinks by reaction of the nucleophilic groups with the electrophilic groups to form the hydrogel.

21. The method any of 1-20 wherein the precursor is water soluble.

22. The method any of 1-21 wherein the hydrogel is formed by combining a first synthetic precursor with a second synthetic precursor.

26. The method of any of 1-22 wherein the therapeutic agent is selected from the group consisting of an antiagngiogenic agent, dexamethasone, nifedipine, a steroid, an inhibitor of vascular endothelial growth factor, a small molecule drug, a protein, a nucleic acid, and a growth factor.

27. The method of any of 1-26 wherein the therapeutic agent is released over a period of time that is at least about three days.

28. The method of any of 1-26 wherein a time at which a cumulative 80% w/w of the therapeutic agent has been released is between about 14 and 180 days. Artisans will immediately appreciate that all ranges and values between the explicitly stated bounds are contemplated, with, e.g., any of the following being available as an upper or lower limit: 0.5, 1, 2, 3, 4, 5, 6 months.

29. The method of any of 1-28 further comprising puncturing a conjunctiva to access the choroid.

30. The method of any of 1-29 wherein the layer is essentially between a choroid and a sclera of the eye, or is substantially in the choroid.

31. The method of any of 1-30 wherein the pathology is a back-of-the eye disease.

32. The method of 31 wherein the back-of-the-eye disease is a selected from the group consisting of wet macular degeneration, dry macular degeneration, diabetic macular edema, cystoid macular edema, and diabetic retinopathy.

33. The method of 31 wherein the back-of-the-eye disease is an ocular disease of a posterior segment of the eye that affects a retina, macula or choroid, with said disease leading to a visual acuity disturbance, loss of sight, or blindness.

34. An injection device for injection into a tissue, for example an eye, comprising a syringe, and a needle, with the syringe comprising a reservoir in fluid communication with an outlet and a piston for expelling contents of the reservoir from the syringe through the outlet.

35. The device of 34 wherein a force applied to the piston (directly or indirectly, e.g., at a plunger connected to the piston) is greater than a force required to move the needle through a sclera and, while that force is applied, the reservoir has a pressure that is less than a pressure required to inject a fluid from the syringe into a sclera and is at least equal to a pressure required to inject a fluid at a choroid.

36. The device of 34 wherein the reservoir, before use, comprises a fluid and has a pressure that is less than a pressure required to inject a fluid from the syringe into a sclera and is at least equal to a pressure required to inject a fluid at a choroid.

37. The device of any of 34-36 further comprising a biasing member, a spring bias member (compressible or extendible), a spring (compressible or extendible), a dashpot, a permanently compressible member, or a reversibly compressible member, or a combination thereof.

38. The device of any of 34-37 wherein the needle comprises a portion that is a biasing member, a spring bias member (compressible or extendible), spring (compressible or extendible), permanently compressible member, reversibly compressible member, or combination thereof. The needle may be designed for example: to deliver a constant force at the needle tip, to compress when a certain threshold of force is applied, or provide an increasing amount of force as compression is increased.

39. The device of any of 34-38 wherein the plunger comprises a portion that is a biasing member, a spring bias member (compressible or extendible), spring (compressible or extendible), permanently compressible member, reversibly compressible member, or combination thereof. The plunger may be designed for example: to deliver a constant force at the needle tip, to compress when a certain threshold of force is applied, or provide an increasing amount of force as compression is increased.

40. The device of any of 34-39 wherein the needle is movable relative to a body (and/or the reservoir) of the syringe. Note that embodiments include a needle that extends a spring and/or a needle that compresses a spring when it moves. Dashpots and so forth are also embodiments.

41. The device of any of 34-40 with a constant force applied at the needle in response to variable forces applied to the device, e.g., at the syringe body or the plunger or the piston.

42. A method of using any of the devices of 34-41 for injecting fluent materials, e.g., precursors that form a hydrogel in situ.

43. The method of 42 with the hydrogel being formed at a choroid. (suprachoroidal or in the choroid).

44. A method of injecting a fluent material (e.g., an aqueous solution, a precursor) at a choroid of an eye comprising providing the device of any of 34-41 and pushing the needle into the eye until a tip of the needle contacts a suprachoroidal space, and allowing the device to deliver contents of the reservoir through the needle.

45. A method of injecting a fluent material at a choroid of an eye comprising providing an injection device comprising a syringe and a needle, with the syringe comprising a reservoir in fluid communication with an outlet and a piston for expelling contents of the reservoir from the syringe through the outlet, forcing the needle into the eye to advance the needle through the sclera, wherein a force applied to move the needle applies a piston force to the piston is greater than a force required to move the needle through the sclera and, while the piston force is applied at the piston, the reservoir has a pressure that is less than a pressure required to inject a fluid from the syringe into a sclera and is at least equal to a pressure required to inject a fluid at a choroid, and stopping the advancement of the needle through the sclera when the fluent material leaves the needle, with the needle being at the choroid.

46. A method of injecting a fluent material at a choroid of an eye comprising providing an injection device comprising a syringe and a needle, with the syringe comprising a reservoir in fluid communication with an outlet and a piston for expelling contents of the reservoir from the syringe through the outlet, wherein the reservoir, before use, comprises the fluent material at a pressure that is less than a pressure required to inject the fluent material from the syringe into a sclera and is at least equal to a pressure required to inject the fluid at a choroid, forcing the needle into the eye through the sclera until the needle is at the choroid, and allowing the pressure of the reservoir to move the fluent material into the eye at the choroid.

47. The method of 45 or 46 comprising a biasing member that provides the piston force to the piston, or translates a force on the device into a force at the piston.

48. The method of 47 further comprising a biasing member that provides the piston force to the piston.

49. A method of injecting a fluent material at a choroid of an eye comprising providing an injection device comprising a syringe and a needle, with the syringe comprising a reservoir in fluid communication with an outlet and a piston for expelling contents of the reservoir from the syringe through the outlet, the device further comprising a biasing member connected with the needle or a plunger, said plunger being connected to the piston, applying a force to the device to advance the needle into the eye through the sclera, with the biasing member translating variations in the force into a constant force.

50. The device of 49 wherein the biasing member is set to compress when a certain threshold of force is applied at the needle tip.

51. A method of injecting a fluent material at a tissue of an eye comprising providing an injection device comprising a double reservoir syringe and a needle, with the syringe comprising a first barrel serving as a reservoir in fluid communication with an outlet and a piston for expelling contents of the reservoir from the syringe through the outlet and a second barrel comprising a biasing member, and applying a force to the device to advance the needle into the tissue, with the biasing member providing a resistance to a distal movement of the device.

52. The injection device described in the method of 51.

53. The method or device of 51 or 52 wherein the bias member comprises a spring bias member (compressible or extendible), spring (compressible or extendible), permanently compressible member, reversibly compressible member, or combination thereof.

54. A use of the device or of the method of any of 1-53 to treat an eye, or a patient, or an ophthalmic pathology.

55. A kit comprising the device of, or components for, a method of any of 1-54. For instance, the device, the syringe, the needle. Further, one or more of: diluent, precursor, gel, or fluent materials for injection. Therapeutic agents may be in the kit.

1. An injection device comprising a syringe and a needle, with the syringe comprising a reservoir in fluid communication with an outlet and a piston for expelling contents of the reservoir from the syringe through the outlet.

2. The device of claim 1 wherein the reservoir, before use, comprises a fluent material at a pressure that is less than a pressure required to inject the fluid from the syringe into a sclera and is at least equal to a pressure required to inject the fluid at a choroid.

3. The device of claim 1 further comprising a biasing member that provides the piston force to the piston.

4. The device of claim 1 wherein a piston force applied to the piston is greater than a force required to move the needle through a sclera and, while the piston force is applied at the piston, the reservoir has a pressure that is less than a pressure required to inject a fluid from the syringe into a sclera and is at least equal to a pressure required to inject a fluid at a choroid.

5. The device of claim 1 wherein the needle has a distal tip and comprises a portion that is a biasing member set to deliver a constant force at the distal tip, set to compress when a certain threshold of force is applied at the needle tip, or set to provide an increasing amount of force as compression of the bias member is increased in response to a force applied at the distal tip.

6. The device of claim 1 wherein the needle has a distal tip and comprising a plunger connected to the piston, wherein the plunger comprises a portion that is a biasing member.

7. The device of claim 6 wherein the biasing member is set to deliver a constant force at the distal tip, to compress when a certain threshold of force is applied to the distal tip, or to provide an increasing amount of force as compression of the biasing member is increased in response to a force applied at the distal tip.

8. The device of claim 1 wherein the needle is movable relative to the syringe.

9. The device of claim 8 wherein the needle is connected to a biasing member, a spring bias member or a dashpot to control movement of the needle.

10. The device of claim 1 wherein a variable force applied to the device is translated into a constant force at the needle.

11. The device of claim 10 wherein a biasing member, a dashpot, a spring bias member, a spring, an extendible spring, a compressible spring, a permanently compressible member, or a reversibly compressible member translates the variable force into the constant force.

12. The device of claim 1 further comprising, in the reservoir, a precursor to form a hydrogel in situ.

13. A method of treating an ophthalmic pathology affecting an eye of a patient comprising forming, in situ, a continuous cohesive layer of covalently-crosslinked hydrogel at a choroid in the eye, wherein the hydrogel comprises a therapeutic agent that is released into the eye to treat the ophthalmic pathology.

14. The method of claim 13 comprising introducing a hydrogel precursor in aqueous solution at the choroid that flows from the site and reacts to form the hydrogel.

15. The method of claim 14 wherein the precursor is a first precursor, further comprising a second precursor in the aqueous solution, with the first precursor and second precursor crosslinking with each other to form the hydrogel.

16. A method of injecting a fluent material at a choroid of an eye comprising
providing an injection device comprising a syringe and a needle, with the syringe comprising a reservoir in fluid communication with an outlet and a piston for expelling contents of the reservoir from the syringe through the outlet,
forcing the needle into the eye to advance the needle through the sclera,
wherein a force applied to move the needle applies a piston force to the piston is greater than a force required to move the needle through the sclera and, while the piston force is applied at the piston, the reservoir has a pressure that is less than a pressure required to inject a fluid from the syringe into a sclera and is at least equal to a pressure required to inject a fluid at a choroid, and
stopping the advancement of the needle through the sclera when the fluent material leaves the needle, with the needle being at the choroid.

17. A method of injecting a fluent material at a choroid of an eye comprising
providing an injection device comprising a syringe and a needle, with the syringe comprising a reservoir in fluid communication with an outlet and a piston for expelling contents of the reservoir from the syringe through the outlet,
wherein the reservoir, before use, comprises the fluent material at a pressure that is less than a pressure required to inject the fluent material from the syringe into a sclera and is at least equal to a pressure required to inject the fluid at a choroid,
forcing the needle into the eye through the sclera until the needle is at the choroid, and
allowing the pressure of the reservoir to move the fluent material into the eye at the choroid.

18. A method of injecting a fluent material at a choroid of an eye comprising
providing an injection device comprising a syringe and a needle, with the syringe comprising a reservoir in fluid communication with an outlet and a piston for expelling contents of the reservoir from the syringe through the outlet,
the device further comprising a biasing member connected with the needle or a plunger, said plunger being connected to the piston,
applying a force to the device to advance the needle into the eye through the sclera,
with the biasing member translating variations in the force into a constant force.

19. The method of claim 18 wherein the biasing member is set to compress when a certain threshold of force is applied at the needle tip.

20. A double reservoir syringe and a needle, with the syringe comprising a first barrel serving as a reservoir in fluid communication with an outlet and a piston for expelling contents of the reservoir from the syringe through the outlet and a second barrel comprising a biasing member, and
applying a force to the device to advance the needle into the tissue, with the biasing member providing a resistance to a distal movement of the device.
